(19) United States
(12) Patent Application Publication
(10) Pub. No.: US 2026/0001948 A1
(43) Pub. Date: Jan. 1, 2026

(54) Targeting the Interplay of Treg Cells and Hepatic Stellate Cells in Liver Fibrosis and Insulin Resistance

(71) Applicant: The Trustees of Columbia University in the City of New York, New York, NY (US)

(72) Inventors: Nicholas ARPAIA, New York, NY (US); Thomas SAVAGE, Bronx, NY (US)

(73) Assignee: The Trustees of Columbia University in the City of New York, New York, NY (US)

(21) Appl. No.: 19/277,868

(22) Filed: Jul. 23, 2025

Related U.S. Application Data
(63) Continuation of application No. PCT/US2024/012638, filed on Jan. 23, 2024.
(60) Provisional application No. 63/440,641, filed on Jan. 23, 2023.

(51) Int. Cl. C07K 16/24; A61K 39/00; A61P 5/50

(52) U.S. Cl. CPC C07K 16/248; A61P 5/50; A61K 2039/505; C07K 2317/24

(57) ABSTRACT

Methods of treating subjects suffering from non-alcoholic steatohepatitis (NASH) by interfering with epidermal growth factor (EGFR) and interleukin 6 (IL-6) production and/or function in hepatic stellate cells.

### CROSS-REFERENCE TO RELATED APPLICATIONS

This is a continuation of International Patent Application No. PCT/US2024/012638 filed on 23 Jan. 2024, which claims the benefit of U.S. Provisional Application No. 63/440,641 filed 23 Jan. 2023, each of which are incorporated herein in their entirety.

### STATEMENT REGARDING FEDERALLY SPONSORED RESEARCH OR DEVELOPMENT

This invention was made with government support under grants AI127847, GM007367, and HL148718 awarded by the National Institutes of Health. The government has certain rights in the invention.

### TECHNICAL FIELD OF THE INVENTION

This disclosure generally relates to the fields of medicine and immunology. More specifically, the disclosure relates to the treatment of subjects suffering from non-alcoholic steatohepatitis (NASH) by interfering with epidermal growth factor (EGFR) and interleukin 6 (IL-6) production and/or function in hepatic stellate cells.

### BACKGROUND OF THE INVENTION

Fibrosis is an end stage disease and a leading cause of morbidity and mortality worldwide, with a paucity of effective therapeutic options. Identifying principles underlying the development of tissue fibrosis may therefore offer new therapeutic targets. Non-alcoholic fatty liver disease (NAFLD) and its progressive form, non-alcoholic steatohepatitis (NASH), currently affect up to 30% of the world population and will soon become the most prevalent cause of chronic liver disease. The progression to NASH involves hepatocyte damage and liver inflammation, with the development of liver fibrosis being the key determinant of patient outcomes. As yet, however, there are no effective pharmacological therapies for NASH or NASH-mediated fibrosis, stemming at least in part from the limited understanding of cellular and molecular mechanisms driving disease progression.

NAFLD and NASH are closely associated with insulin resistance. Over half of patients with type 2 diabetes have fatty liver disease, and more than 40% of patients with NASH have type 2 diabetes, increasing proportionally with greater severity of NASH and fibrosis. Insulin resistance contributes to the development of NAFLD and its progression to NASH, but the role for NASH in promoting insulin resistance—and the molecular and cellular interactions underlying NASH induced metabolic decline—is poorly understood and thus therapeutically unexplored.

Furthermore, hepatic gluconeogenesis is also a key therapeutic target in type 2 diabetes, yet existing therapeutics target either insulin resistance or NASH and NASH-mediated liver fibrosis, but not both. Additionally, hepatic stellate cells—liver-resident fibroblasts—are central to liver fibrosis and interplay with hepatocytes via cell-cell contact and soluble mediators that support hepatocyte development and homeostasis. However, few studies have focused on the interplay between hepatic stellate cells and hepatocytes in the setting of metabolic disease and, more specifically, whether activated hepatic stellate cells produce mediators that promote insulin resistance in hepatocytes. In metabolic disease, pro-inflammatory cytokines such as IL-6 from adipose tissue macrophages are reported to circulate systemically and promote hepatocyte insulin resistance. However, whether cells in closer proximity (e.g. hepatic stellate cells)—which can likely deliver higher local concentrations—produce mediators that promote hepatocyte insulin resistance is unclear.

Regulatory T (Treg) cells are a specialized T cell subset that have been studied for their protective roles in tissue homeostasis by limiting inflammation and producing molecules that promote repair. In studies to date, Treg cell activity is considered a key component of the adaptive response to tissue damage, and necessary to support effective tissue repair and restoration. Beneficial Treg cell functions are apparent in the uncontrolled inflammation and fatal tissue damage observed in Foxp3-mice, a phenotype that can be rescued by transfer of T cells expressing Foxp3. Furthermore, Treg cell depletion results in increased insulin resistance2 and worsened liver injury and fibrosis. These studies established a paradigm in which the present inventors view Treg cells as immunosuppressive and tissue protective—properties of interest as potential therapeutic targets in numerous diseases—and have led to investigations of the mediators such as cytokines and molecular features that endow critical Treg cell functions. In addition to immunosuppressive mediators, recent work has demonstrated that Treg cells express epithelial growth factors that contribute to tissue protection. Treg cell production of amphiregulin (Areg), a low-affinity ligand for the Epidermal Growth Factor Receptor (EGFR)-family, is critical to repair following acute muscle, brain, and lung injury.

In these settings, Treg cell-derived Areg is notably independent of Treg cell-mediated immunosuppression and signals to tissue-resident mesenchymal cells and stem cells—not immune cells—to mediate its function. Together, these studies suggest that Treg cells protect from epithelial cell injury by both interacting with immune cells to suppress inflammation and by producing epithelial growth factors that signal to non-immune cells. As described hereinafter, Areg from Treg cells plays a maladaptive role, driving liver fibrosis and promoting NASH-dependent glucose intolerance. These findings reveal potential new therapeutic avenues for treating fibrotic disease by targeting interactions between Treg cells and tissue-resident fibroblasts by using agents that interfere with the production and/or function of EGFR and IL-6 in animal models and other subjects with NASH.

### BRIEF SUMMARY OF THE INVENTION

The present disclosure relates to methods of treating subjects suffering from non-alcoholic steatohepatitis (NASH) by administering one or more epidermal growth factor (EGFR) inhibitors, interleukin 6 (IL-6) receptor inhibitors, or IL-6 inhibitors.

In some embodiments, the disclosed methods comprise administering to a subject a therapeutically effective amount of an adeno-associated virus (AAV) vector that results in (a) a mutation or deletion of a gene encoding an epidermal growth factor receptor expressed in hepatic stellate cells in the subject; or (b) a mutation or deletion of a gene encoding an interleukin-6 receptor expressed in hepatocytes in the subject; or (c) a mutation or deletion of a gene encoding interleukin-6 expressed in hepatic stellate cells in the subject.

In some embodiments, the disclosed methods comprise administering to a subject a therapeutically effective amount of one or more anti-EGFR inhibitors described herein, or a pharmaceutical composition comprising one or more (e.g., two or three) anti-EGFR inhibitors described herein in combination with another therapy to treat NASH or its symptoms. In some embodiments, the anti-EGFR inhibitor is a small molecule tyrosine kinase inhibitor (TKI). In some embodiments, the anti-EGFR inhibitor is an antibody.

In some embodiments, the disclosed methods comprise administering to a subject a therapeutically effective amount of one or more anti-IL-6 receptor inhibitors described herein, or a pharmaceutical composition comprising one or more (e.g., two or three) anti-IL-6 receptor inhibitors described herein in combination with another therapy to treat NASH or its symptoms. In some embodiments, the anti-IL-6 receptor inhibitor is an antibody.

In some embodiments, the disclosed methods comprise administering to a subject a therapeutically effective amount of one or more anti-IL-6 inhibitors described herein, or a pharmaceutical composition comprising one or more (e.g., two or three) anti-IL-6 inhibitors described herein in combination with another therapy to treat NASH or its symptoms. In some embodiments, the anti-IL-6 inhibitor is a small molecule kinase inhibitor. In some embodiments, the anti-IL-6 inhibitor is an antibody.

In some embodiments, the inhibitors described herein may be administered to a subject or delivered to a tumor in the form of a pharmaceutical composition, which may comprise one or more pharmaceutically acceptable carriers, diluents, or excipients.

The present disclosure also relates to articles of manufacture useful for treating NASH. In some embodiments, the articles of manufacture comprise a container comprising an inhibitor described herein, or pharmaceutical compositions comprising the same, as well as instructional materials for using the same to treat NASH. In some embodiments, the articles of manufacture are part of a kit.

The foregoing is a summary and thus contains, by necessity, simplifications, generalizations, and omissions of detail; consequently, those skilled in the art will appreciate that the summary is illustrative only and is not intended to be in any way limiting. Other aspects, features, and advantages of the methods, compositions and/or devices and/or other subject matter described herein will become apparent in the teachings set forth herein. The summary is provided to introduce a selection of concepts in a simplified form that are further described below in the Detailed Description of the Invention. This summary is not intended to identify key features or essential features of the claimed subject matter, nor is it intended to be used as an aid in determining the scope of the claimed subject matter.

### BRIEF DESCRIPTION OF THE SEVERAL VIEWS OF THE DRAWINGS

FIGS. 1A-1K show how Treg cell-derived amphiregulin promotes liver fibrosis. (A) Flow cytometric analysis of Areg protein expression by the indicated mouse T cell subsets. Tconv.: TCRβ⁺CD4⁺Foxp3⁻. Data are representative of 2 independent experiments, each with n=4 mice. (B-D) Liver Treg cell frequency and number, and number of liver Areg-producing Treg cells, as assessed by flow cytometric analysis from mice fed normal chow or FPC diet. n≥3 mice per group. Data are representative of 2 independent experiments. (E-F) Quantification of Treg cell staining in human healthy or NASH liver specimens. n=9-10 per group. (G-J) Sirius Red staining of liver sections from mice lacking Treg cell-derived Areg (Areg^{Fl}/FlFoxp3^{YFP-cre}) or littermate controls (Areg^{Fl/Fl}) fed (G-H) FPC diet for 16 weeks or (I-J) the CDAA-HFD. n≥6 mice per group. Data are representative of 2-4 independent experiments. (K) Expression of indicated genes in liver tissue as assessed by RT-qPCR of Areg^{Fl}/FlFoxp3^{YFP-cre} or littermate Areg^{Fl/Fl} mice fed the FPC diet for 16 weeks. Data representative of 4 independent experiments. (B-F, H, J-K) two-sided Student's t-test: *p<0.05, **p<0.01, ***p<0.001. Data are expressed as mean±s.e.m

FIGS. 2A-2M illustrate that liver Treg cells have a unique transcriptional profile in fibrosis. (A) Uniform Manifold Approximation and Projection (UMAP) plot of single cell RNA-Seq analysis of Treg cells from liver, spleen and VAT of mice fed normal chow or CDAA-HFD. (B) Expression of indicated gene as displayed by a feature plot among all Treg cells. (C-D) Differentially expressed genes between Treg cells at steady state in (C) the liver and spleen and (D) liver and VAT. (E) Overview of ARACNe-AP and msVIPER analyses to score transcription factor activity in Treg cells of each sample. (F) Differential transcription activity in normal chow Treg cells from liver compared to spleen Treg cells, as analyzed by msVIPER. (G) Transcription factors with increased activity in normal chow Treg cells from liver versus spleen that also had increased activity in VAT Treg cells compared to spleen (top) or had increased activity in liver versus spleen Treg cells but not in VAT versus spleen Treg cells (bottom). (H-I) Differential gene expression and representative gene ontology (GO) terms between liver Treg cells from NASH compared to normal chow. (J) Differential transcription activity in liver Treg cells from normal chow compared to NASH, as analyzed by msVIPER. (K) Frequency of liver Treg cells in each cluster per mouse from normal chow or NASH. (L) Proportion of Treg cell TCR clonotypes with each level of clonal expansion among liver Treg cells from Ccr8 cluster. (M) Normalized entropy score of Treg cell TCR clonotypes from liver Ccr8 cluster.

FIGS. 3A-3K show that Treg cell-derived Areg directly activates hepatic stellate cells to promote liver fibrosis in NASH. (A-D) Hepatic stellate cell lineage trace mice (Rosa26R^{tdTomato}Lrat-Cre) were fed the (A-B) FPC diet for 16 weeks or (C-D) CDAA-HFD for 8 weeks. (A, C) Treg cell (CD4⁺Foxp3⁺) and CD4⁺Foxp3⁻ T cell localization relative to hepatic stellate cells (tdTomato⁺) was assessed by confocal imaging analysis, with (B, D) quantification of the distance of Treg cells (Foxp3⁺ cells) and CD4⁺Foxp3⁻Tconv (CD4⁺ Tconv) to the nearest hepatic stellate cell across multiple sections stained from n=5 mice. Representative images (A, C), yellow arrowheads indicate Foxp3⁺ cells. (E-F) RNA-Seq analysis of purified quiescent hepatic stellate cells left untreated, or treated with recombinant murine AREG (rmAREG) in vitro. n=5 samples per group. (E) Selected genes significantly upregulated following Areg treatment and (F) representative gene ontology terms significantly enriched by Areg treatment. (G-J) Sirius Red staining of livers from hepatic stellate cell-specific Egfr conditional knockout (Egfr^{Fl/Fl}Lrat-Cre) and littermate control (Egfr^{Fl/Fl}) mice fed (G-H) FPC diet for 16 weeks (n≥7 mice per group) or (I-J) CDAA-HFD (n≥4 mice per group). Data representative of 2-3 independent experiments. (K) Expression of indicated genes in liver tissue as assessed by RT-qPCR of Egfr^{Fl/Fl}Lrat-Cre or littermate Egfr^{Fl/Fl} mice fed the FPC diet for 16 weeks. Data representative of 3 independent experiments. (B, D, F, H, K): two-sided Student's t-test. **p<0.01, ***p<0.001. Data are expressed as mean±s.e.m.

FIGS. 4A-4G show that Treg cell-derived Areg promotes the activation of quiescent hepatic stellate cells in NASH. (A) UMAP plot displaying clusters of cells in single cell RNA-Seq of liver cells enriched for hepatic stellate cells from Areg^{Fl/Fl} or Areg^{Fl/Fl}Foxp3^{YFP-cre} mice (n=2 mice per genotype) fed the CDAA-HFD. (B) Feature plots displaying expression of indicated genes across cell clusters. (C) For each indicated hepatic stellate cell cluster, differential gene expression analysis between cells from Areg^{Fl/Fl} (wildtype, ‘WT’) compared to Areg^{Fl/Fl}Foxp3^{YFP-cre} (conditional knockout, ‘cKO’) mice. Number of significantly up- and down-regulated genes (average Log₂ fold change >0 or <0, respectively; P_{adj}<0.05) indicated. (D) Representative gene ontology terms with differential enrichment between Areg^{Fl/Fl} (‘WT’) compared to Areg^{Fl/Fl}Foxp3^{YFP-cre} Treg cell-derived Areg conditional knockout (‘cKO’) Fcna-expressing hepatic stellate cells (top) and Mmp2-hepatic stellate cells (bottom). Red: enriched in WT cells; blue: enriched in cKO cells. (E) Overview of ARACNe-AP and VIPER analyses to determine relative transcription factor activity in hepatic stellate cells. compared to Areg^{Fl/Fl}Foxp3^{YFP-cre} (‘cKO’) mice, with fibrosis activity score indicated by color. Fibrosis activity score based on output from ARACNe-AP. (G) Volcano plot of transcription factor activity of hepatic stellate cells from Areg^{Fl/Fl} (‘WT’) compared to Areg^{Fl/Fl}Foxp3^{YFP-cre} (‘cKO’) mice. Fibrosis activity score for each transcription factor indicated by color of label.

FIGS. 5A-5F show Treg cell-derived Areg promotes glucose intolerance in a NASH dependent manner through EGFR-signaling on hepatic stellate cells. (A-B) Glucose tolerance tests (GTT) in (A) littermate Areg^{Fl/Fl} (black) and Areg^{Fl/Fl}Foxp3^{YFP-cre} (blue) and (B) littermate Egfr^{Fl/Fl} (black) and Egfr^{Fl/Fl} Lrat-Cre (light blue) mice fed NASH- each with n>4 mice per group. (C-D) GTT of (C) littermate Areg^{Fl/Fl} (black) and Areg^{Fl/Fl}Foxp3^{YFP-cre} (blue) and (D) littermate Egfr^{Fl/Fl} (black) and Egfr^{Fl/Fl}Lrat-Cre (light blue) mice fed a HFD that induces insulin resistance without NASH. Data are representative of 2-3 independent experiments, each with n≥4 mice per group. (E) GTT of mice with hepatocyte-specific Egfr deletion (Egfr^{Fl/Fl}-AAV8-TBG Cre) or littermate controls (Egfr^{Fl/Fl}-AAV8-TBG-NULL) fed the FPC diet for 16 weeks. Adult Egfr^{Fl/Fl} mice were administered AAV8-TBG-Cre or AAV8-TBG-null viral particles 1 week prior to beginning diet experiment. n=8 mice per group. Data are representative of 2 independent experiments. (F) GTT of mice with hepatocyte-specific Egfr deletion or littermate controls (as in (E)) fed a HFD that does not induce NASH for 8 weeks. n≥5 mice per group. Data are representative of 2 independent experiments. (A-F): 2-way ANOVA with Holm Sidak post-hoc test. (A-F): two-sided Student's t-test. *p<0.05, **p<0.01, ***p<0.001, ****p<0.0001. ns: not significant. Data are expressed as mean±s.e.m.

FIGS. 6A-6N illustrate how Treg cell-derived Areg promotes hepatocyte gluconeogenesis in NASH through hepatic stellate cell-derived IL-6. (A) Blood glucose levels following 16-hour fasting in littermate Areg^{Fl/Fl} (black) and Areg^{Fl/Fl}Foxp3^{YFP-cre} (blue) mice fed (left) FPC diet for 16 weeks or (right) HFD for 8 weeks. Data are representative of 3-4 independent experiments, each with n≥4 mice per group. (B) Pyruvate tolerance test of littermate Areg^{Fl/Fl} (black) and Areg^{Fl/Fl}Foxp3^{YFP-cre} (blue) mice fed the FPC diet for 16 weeks. Data are representative of 3 independent experiments, each with n≥4 mice per group. (C) Blood glucose levels following a 16-hour fast in littermate Egfr^{Fl/Fl} and Egfr^{Fl/Fl}Lrat-Cre mice fed (left) FPC diet for 16 weeks or (right) HFD for 8 weeks. Data are representative of 2-3 independent experiments, each with n≥4 mice per group. (D) Pyruvate tolerance test in mice with hepatic stellate cell-specific Egfr deletion or littermate controls on the FPC diet for 16 weeks. n≥7 mice per group. Data are representative of 3 independent experiments. (E-G) Basal (black) and cAMP/dexamethasone-induced (cAMP+dex; red) glucose production by in vitro-cultured primary mouse hepatocytes. (E) Hepatocytes plated in wells coated with indicated collagen densities. Data are representative of 5 independent experiments. (F) Hepatocytes treated for 18 hours with Medium 199 alone (with or without the addition of rmAREG; “cell-free media”), or treated with conditioned Medium 199 harvested following 23-hour culture of hepatic stellate cells with or without rmAREG. Data are representative of 3 independent experiments. (G) Hepatocytes treated for 18 hours with indicated recombinant protein. Data are representative of 3-5 independent experiments. (H-I) Expression of Il6 in bulk liver tissue of mice on the FPC diet for 16 weeks. (H) Comparison of littermate Areg^{Fl/Fl} and Areg^{Fl/Fl}Foxp3^{YFP-cre} mice. (I) Comparison of littermate Egfr^{Fl/Fl} and Egfr^{Fl/Fl}Lrat-Cre mice. Data are representative of 2-4 independent experiments, each with n≥6 mice per group. (J-K) GTT of littermate Areg^{Fl/Fl} (black) and Areg^{Fl/Fl}Foxp3^{YFP-cre} (blue) mice fed FPC diet for 16 weeks and treated with (J) isotype or (K) IL-6R blocking antibody 48 hours prior to fasting for GTT (see methods). Data are representative of 3 independent experiments, each with n≥4 mice per group. (L-M) GTT of littermate Egfr^{Fl/Fl} and Egfr^{Fl/Fl}Lrat-Cre mice fed FPC diet for 16 weeks and treated with (L) isotype or (M) IL-6R blocking antibody 48 hours prior to fasting for GTT (see methods). Data are representative of 2 independent experiments, each with n≥5 mice per group. (N) GTT of littermate Il6^{Fl/Fl} and Il6^{Fl/Fl} Lrat-Cre mice fed FPC diet for 16 weeks. (A-D, H-N): two-sided Student's t-test. (B, D-G, J-M): 2-way ANOVA with Holm Sidak post-hoc test. *p<0.05, **p<0.01, ***p<0.001. ns: not significant. Data ar expressed as mean±s.e.m.

### DETAILED DESCRIPTION OF THE INVENTION

While the present invention may be embodied in many different forms, disclosed herein are specific illustrative embodiments thereof that exemplify the principles of the invention. It should be emphasized that the present invention is not limited to the specific embodiments illustrated. Moreover, any section headings used herein are for organizational purposes only and are not to be construed as limiting the subject matter described.

Unless otherwise defined herein, scientific, and technical terms used in connection with the present invention shall have the meanings that are commonly understood by those of ordinary skill in the art. Further, unless otherwise required by context, singular terms shall include pluralities and plural terms shall include the singular. More specifically, as used in this specification and the appended claims, the singular forms “a,” “an” and “the” include plural referents unless the context clearly dictates otherwise. Thus, for example, reference to “a protein” includes a plurality of proteins; reference to “a cell” includes mixtures of cells, and the like.

In addition, ranges provided in the specification and appended claims include both end points and all points between the end points. Therefore, a range of 1.0 to 2.0 includes 1.0, 2.0, and all points between 1.0 and 2.0.

The term “about” as used herein when referring to a measurable value such as an amount, a temporal duration, and the like, is meant to encompass variations of .+−.20%,.+−.10%,.+−.5%,.+−.1%, or .+−.0.1% from the specified value, as such variations are appropriate to perform the disclosed methods.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or lists of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of “consisting essentially of,” when used in the claims, shall have its ordinary meaning as used in the field of patent law.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively.

Generally, nomenclature used in connection with, and techniques of, cell and tissue culture, molecular biology, immunology, microbiology, genetics and protein and nucleic acid chemistry and hybridization described herein are those well-known and commonly used in the art. The methods and techniques of the present invention are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art, or as described herein. The nomenclature used in connection with, and the laboratory procedures and techniques of, analytical chemistry, synthetic organic chemistry, and medicinal and pharmaceutical chemistry described herein are those well-known and commonly used in the art.

The inventions described herein relate to methods of treating subjects suffering from non-alcoholic steatohepatitis (NASH) by administering one or more epidermal growth factor (EGFR) inhibitors, interleukin 6 (IL-6) receptor inhibitors, or IL-6 inhibitors as described hereinbelow.

In some embodiments the instant methods comprise administering to a subject a therapeutically effective amount of an adeno-associated virus (AAV) vector comprising a nucleic acid that results in (a) a mutation or deletion of a gene encoding an epidermal growth factor receptor expressed in hepatic stellate cells in the subject; or (b) a mutation or deletion of a gene encoding an interleukin-6 receptor expressed in hepatocytes in the subject; or (c) a mutation or deletion of a gene encoding interleukin-6 expressed in hepatic stellate cells in the subject. Such methods can be accomplished using, e.g., Clustered Regularly-Interspaced Short Palindromic Repeats (CRISPR)/Cas9-mediated gene therapy strategies known in the art. Likewise, mutation strategies to alter protein function, e.g., to interfere with receptor-ligand interactions, are also known in the art.

A variety of gene therapies using AAV vectors are known in the art. For example, nadofaragene firadenovec (ADSTILADRIN®), which is presently used to treat bladder cancer by delivering human interferon alfa-2b cDNA into the bladder epithelium; delandistrogene-moxeparvovec (ELEVIDYS®), which is presently used to treat certain types of Duchenne muscular dystrophy by delivering a micro dystrophin gene; etranacogene dezaparvovec (HEMGENIX®), which is presently used to treat hemophilia B by delivering a Factor IX gene; voretigene neparvovec (LUXTURNA®), which is presently used to treat Leber's congenital amaurosis; valoctocogene roxaparvovec (ROCTAVIAN®), which is presently used to treat hemophilia A by delivering a Factor VIII gene; and onasemnogene abeparvovec (ZOLGENSMA®), which is presently used to treat spinal muscular atrophy.

In some embodiments, the instant methods comprise administering to a subject a therapeutically effective amount of one or more anti-EGFR inhibitors described herein, or a pharmaceutical composition comprising one or more (e.g., two or three) anti-EGFR inhibitors described herein in combination with another therapy to treat NASH or its symptoms.

In some embodiments, the anti-EGFR inhibitor is a small molecule tyrosine kinase inhibitor (TKI). Exemplary embodiments of such inhibitors include TKI such as afatinib (GILOTRIF®), dacomitinib (VIZIMPRO®), neratinib (NERLYNX®), osimertinib (TAGRISSO®), brigatinib (ALUNBRIG®), erlotinib (TARCEVA®), geftinib (IRESSA®), lapatinib (TYKERB®), mobocertinib (EXKIVITY®), tivozanib (FOTIVDA®), pemetrexed (PEMFEXY®), and vandetanib (CAPRELSA®). The foregoing TKI are presently used for the treatment of a variety of cancers, with or without other chemotherapeutics, depending upon the particulars of each TKI.

In some embodiments, the anti-EGFR inhibitor is an antibody. In some embodiments, the anti-EGFR antibody is cetuximab (ERBITUX®), which is presently used for the treatment of metastatic colorectal cancer and head and neck cancer. In other embodiments, the anti-EGFR antibody is panitumumab (VECTIBIX®), which is presently used for the treatment of metastatic colorectal cancer. In other embodiments, the anti-EGFR antibody is necitumumab (PORTRAZZA®), which is presently used for the treatment of squamous non-small-cell lung carcinoma.

In some embodiments, the instant methods comprise administering to a subject a therapeutically effective amount of one or more anti-IL-6 receptor inhibitors described herein, or a pharmaceutical composition comprising one or more (e.g., two or three) anti-IL-6 receptor inhibitors described herein in combination with another therapy to treat NASH or its symptoms.

In some embodiments, the anti-IL-6 receptor inhibitor is an antibody. In some embodiments, the anti-IL-6 antibody is sarilumab (KEVZARA®), which is presently used for the treatment of moderate to severe rheumatoid arthritis. In some embodiments, the anti-IL-6 antibody is tocilizumab (ACTEMRA®), which is presently used for the treatment of moderate to severe rheumatoid arthritis. In some embodiments, the anti-IL-6 antibody is satralizumab (ENSPRYNG®), which is presently used to treat neuromyelitis optica spectrum disorder (NMOSD) in adult patients who are anti-aquaporin-4 (AQP4) antibody positive.

In some embodiments, the instant methods comprise administering to a subject a therapeutically effective amount of one or more anti-IL-6 inhibitors described herein, or a pharmaceutical composition comprising one or more (e.g., two or three) anti-IL-6 inhibitors described herein in combination with another therapy to treat NASH or its symptoms.

In some embodiments, the anti-IL-6 inhibitor is a small molecule kinase inhibitor such as tofacitinib (XELJANZ®), which is presently used to treat rheumatoid arthritis and moderate to severe ulcerative colitis (UC). In some embodiments, the anti-IL-6 inhibitor is an antibody. In some embodiments, the anti-IL-6 antibody is siltuximab (SYLVANT®), which is presently used for the treatment of Multicentric Castleman's Disease (MCD).

As used herein, “treatment” or “treating” or “treat” refers to all processes wherein there may be a slowing, interrupting, arresting, controlling, stopping, alleviating, or ameliorating symptoms or complications or reversing of the progression of NASH, but does not necessarily indicate a total elimination of all symptoms.

Depending on the form of inhibitor, mode of intended delivery, and numerous other variables, inhibitors disclosed herein may be formulated as desired using art recognized techniques. Various pharmaceutically acceptable carriers, which include vehicles, adjuvants, and diluents, are readily available from numerous commercial sources. Moreover, an assortment of pharmaceutically acceptable auxiliary substances, such as pH adjusting and buffering agents, tonicity adjusting agents, stabilizers, wetting agents, and the like, are also available. Certain non-limiting exemplary carriers include saline, buffered saline, dextrose, water, glycerol, ethanol, and combinations thereof.

In some embodiments, inhibitors may be administered to a subject neat or with a minimum of additional components. In other embodiments, inhibitors may be formulated to contain suitable pharmaceutically acceptable carriers comprising excipients and auxiliaries that are well known in the art and are relatively inert substances that facilitate administration or which aid processing of the active compounds into preparations that are pharmaceutically optimized for delivery. For example, an excipient can give form or consistency or act as a diluent to improve the pharmacokinetics of the antibody. Suitable excipients include but are not limited to stabilizing agents, wetting, and emulsifying agents, salts for varying osmolality, encapsulating agents, buffers, and skin penetration enhancers.

In general, inhibitors disclosed herein may be administered in vivo to a subject in need thereof, by various routes, including, but not limited to, oral, intravenous, intra-arterial, subcutaneous, parenteral, intranasal, intramuscular, intracardiac, intraventricular, intratracheal, buccal, rectal, intraperitoneal, intradermal, topical, transdermal, and intrathecal, or otherwise by implantation or inhalation. Compositions may be formulated into preparations in solid, semi-solid, liquid, or gaseous forms; including, but not limited to, tablets, capsules, powders, granules, ointments, solutions, suppositories, enemas, injections, inhalants, and aerosols. The appropriate formulation and route of administration may be selected according to the intended application and therapeutic regimen.

Similarly, the particular dosage regimen, i.e., dose, timing, and repetition, will depend on the particular individual and that individual's medical history. Empirical considerations such as pharmacokinetics (e.g., half-life, clearance rate, etc.) will contribute to the determination of the dosage. Frequency of administration may be determined and adjusted over the course of therapy. Alternatively, sustained continuous release formulations of a subject therapeutic composition may be appropriate. Various formulations and devices for achieving sustained release are known in the art.

Pharmaceutical compositions are administered in therapeutically effective amount in order to treat NASH in a subject. As used herein, the term “therapeutically effective amount” means that amount of an inhibitor or pharmaceutical composition comprising the same that will elicit the biological or medical response in the subject that is sought by a medical doctor or other clinician. In particular, with regard to contemplated sex hormone-related disorders, a “therapeutically effective amount” is intended to include an amount sufficient to address one or more symptoms commonly observed in NASH. In some embodiments, a therapeutically effective amount of an inhibitor or pharmaceutical composition comprising the same has a beneficial effect but does not cure NASH. In certain embodiments, therapy may encompass the administration of multiple doses of an inhibitor or pharmaceutical composition comprising the same at a certain frequency to achieve a therapeutic effect.

A therapeutically effective amount is typically dependent on the weight of the subject being treated, his or her physical condition, the extensiveness of the condition to be treated, and the age of the subject being treated. For example, various antibodies described hereinabove may be administered in an amount in the range of about 10 ng/kg body weight to about 1000 mg/kg body weight per dose. In certain embodiments, antibodies may be administered in an amount in the range of about 50 μg/kg body weight to about 500 mg/kg body weight per dose. In other embodiments, antibodies may be administered in an amount in the range of about 100 μg/kg body weight to about 1000 mg/kg body weight per dose. In other embodiments, antibodies may be administered in an amount in the range of about 100 μg/kg body weight to about 200 mg/kg body weight per dose. In other embodiments, antibodies may be administered in an amount in the range of about 0.5 mg/kg body weight to about 20 mg/kg body weight per dose. In other embodiments, antibodies may be administered in a dose of at least about 100 μg/kg body weight, at least about 250 μg/kg body weight, at least about 750 μg/kg body weight, at least about 3 mg/kg body weight, at least about 5 mg/kg body weight, or at least about 10 mg/kg body weight.

Other dosing regimens may be predicated on Body Surface Area (BSA) calculations. As is well known in the art, a subject's BSA is calculated using the subject's height and weight and provides a measure of a subject's size as represented by the surface area of his or her body. For example, various antibodies described hereinabove are administered in dosages from 10 mg/m² to 800 mg/m². In other embodiments, antibodies are administered in dosages from 50 mg/m² to 500 mg/m² and even more preferably at dosages of 100 mg/m², 150 mg/m², 200 mg/m², 250 mg/m², 300 mg/m², 350 mg/m², 400 mg/m² or 450 mg/m². Escalation for an individual subject can occur at the discretion of a clinician in the absence of any clinically significant occurrence that the clinician might reasonably believe would present an undue safety risk for the subject, such as, for example, infusion reactions, acute anaphylaxis, and serum sickness.

Inhibitors disclosed herein are usually administered to a subject on multiple occasions in accordance with regimens known in the art. In some instances, two or more antibodies with different binding specificities may be administered simultaneously, in which case the dosage of each antibody administered falls within the ranges indicated. Intervals between single dosages can be weekly, monthly, or yearly. Intervals can also be irregular depending upon levels of the inhibitor in the blood and other clinical indicia. In some methods, the dosage is adjusted to achieve a plasma concentration of about 1-1000 μg/mL or about 25-300 μg/mL. Alternatively, inhibitors can be administered as a sustained release formulation, in which case less frequent administration is required.

Dosage and frequency will vary depending on the half-life of the inhibitor in the subject. The duration of a therapeutic regimen depends on a variety of factors that are readily appreciated by one of skill in the art. A clinician can observe the therapy's effects closely and make any adjustments as needed. When inhibitors are used in combination with another inhibitor or another therapy, the two or more therapeutic inhibitors may be administered simultaneously or sequentially in any order. For example, a combination therapy may be performed by administering a first inhibitor prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concurrently with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) administering a second inhibitor. The dosage, frequency, and mode of administration of each component of a combination therapy can be controlled independently. For example, one inhibitor may be administered orally three times per day, while the second inhibitor may be administered intravenously once per day. Combination therapy may be given in on-and-off cycles that include rest periods. The compounds may also be admixed or otherwise formulated together such that one administration delivers both inhibitors. In this case, each inhibitor is generally present in an amount of 1-95% by weight of the total weight of the composition. Alternatively, inhibitors can be formulated separately and in individual dosage amounts. Combinations of inhibitors for treatment can be provided as components of a pharmaceutical pack.

Preferably, combination therapies elicit a synergistic therapeutic effect, i.e., an effect greater than the sum of their individual effects or therapeutic outcomes, such as those described above. For example, a synergistic therapeutic effect may be an effect of at least about two-fold greater than sum of the therapeutic effects elicited by the single agents of a given combination, or at least about five-fold greater, or at least about ten-fold greater, or at least about twenty-fold greater, or at least about fifty-fold greater, or at least about one hundred-fold greater. A synergistic therapeutic effect may also be observed as an increase in therapeutic effect of at least 10% compared to the sum of the therapeutic effects elicited by the single agents of a given combination, or at least 20%, or at least 30%, or at least 40%, or at least 50%, or at least 60%, or at least 70%, or at least 80%, or at least 90%, or at least 100%, or more. A synergistic effect is also an effect that permits reduced dosing of therapeutic agents when they are used in combination.

The inventions disclosed herein also encompass kits comprising one or more containers and comprising one or more doses of an inhibitor disclosed herein. In certain embodiments, a unit dosage is provided wherein the unit dosage contains a predetermined amount of a composition comprising, for example, a kinase inhibitor or antibody disclosed herein, with or without one or more additional agents. For other embodiments, such a unit dosage is supplied in single-use prefilled syringe for injection. In still other embodiments, the composition contained in the unit dosage may comprise saline, sucrose, or the like; a buffer, such as phosphate, or the like; and/or be formulated within a stable and effective pH range. Alternatively, in certain embodiments, the composition may be provided as a lyophilized powder that may be reconstituted upon addition of an appropriate liquid, for example, sterile water. In certain preferred embodiments, the composition comprises one or more substances that inhibit protein aggregation, including, but not limited to, sucrose and arginine. Any label on, or associated with, the container(s) indicates that the enclosed composition is used for diagnosis or treatment.

The present invention also provides kits for producing single-dose or multi-dose administration units of an inhibitor disclosed herein and, optionally, one or more other diagnostic or therapeutic agents. The kit comprises a container and a label or insert on or associated with the container. Suitable containers include, e.g., bottles, vials, and syringes. The containers may be formed from a variety of materials such as glass or plastic. The container holds a composition that is effective for treating the condition and may have a sterile access port (for example the container may be an intravenous solution bag or a vial having a stopper pierceable by a hypodermic injection needle). Such kits will generally contain in a suitable container a pharmaceutically acceptable formulation of the inhibitor and, optionally, one or more other diagnostic or therapeutic agents in the same or different containers. The kits may also contain other pharmaceutically acceptable formulations, either for diagnosis or combined therapy. Such kits may also provide appropriate reagents to conjugate an inhibitor with the other diagnostic or therapeutic agent(s).

More specifically the kits may have a single container that contains the inhibitor with or without additional components, or they may have distinct containers for each desired agent. Where combined therapeutics are provided for conjugation, a single solution may be pre-mixed, either in a molar equivalent combination, or with one component in excess of the other. Alternatively, the inhibitor and any optional diagnostic or therapeutic agent of the kit may be maintained separately within distinct containers prior to administration to a subject. The kits may also comprise a second/third container means for containing a sterile, pharmaceutically acceptable buffer or other diluent such as water for injection (WFI), phosphate-buffered saline (PBS), Ringer's solution, and dextrose solution. When the components of the kit are provided in one or more liquid solutions, the liquid solution is preferably an aqueous solution, with a sterile aqueous solution being particularly preferred. However, the components of the kit may be provided as dried powder(s). When reagents or components are provided as a dry powder, the powder can be reconstituted by the addition of a suitable solvent. It is envisioned that the solvent may also be provided in another container.

As described above, the kits may also contain a means by which to administer the inhibitor and any optional components to the subject, e.g., one or more needles or syringes, or even an eye dropper, pipette, or other such like apparatus, from which the inhibitor or a formulation comprising the same may be injected or introduced into the subject. Such kits will also typically include a means for containing the vials, or such like, and other component in close confinement for commercial sale, such as, e.g., injection or blow-molded plastic containers into which the desired vials and other apparatus are placed and retained. Any label or package insert indicates that the inhibitor is used for treating NASH.

### EXAMPLES

The following examples have been included to illustrate aspects of the inventions disclosed herein. In light of the present disclosure and the general level of skill in the art, those of skill appreciate that the following examples are intended to be exemplary only and that numerous changes, modifications, and alterations may be employed without departing from the scope of the disclosure.

### Example 1

### Mice

Foxp3^{GFP}, Areg^{Fl/Fl}, Egfr^{Fl/Fl}, Lyz2^{wt/cre} (JAX stock #004781), Areg^{Fl/Fl}Foxp3^{YFP-cre}, Areg^{Fl/Fl}CD4-Cre, Rosa26R^{tdTomato} (JAX stock #007914), Vav1-Cre (JAX stock #008610) and Lrat-Cre transgenic mice have been previously described. Areg^{Fl/F}lLyz2^{wt/cre}, Areg^{Fl/Fl}lVav1-Cre, Egfr^{Fl/Fl}Lrat-Cre and Rosa26R^{tdTomato}Lrat-Cre were generated by breeding Areg^{Fl/Fl}, Egfr^{Fl/Fl} or Rosa26R^{tdTomato} mice as applicable with indicated Cre recombinase expressing mice. For some experiments, C57BL/6 male mice—either 10 weeks old or retired breeders-were purchased from Charles River. Mice were screened for maintenance of the C57BL/6N Nnt allele. Areg^{Fl/Fl}Foxp3^{YFP-cre} and Areg^{Fl/Fl} mice were screened for germline deletion, and mice with germline deletion were excluded from experimental cohorts. To delete Egfr on hepatocytes, 1×10¹¹ viral of AAV.TBG.PI.Cre.rBG (AAV8-TBG-Cre) or control particles AAV.TBG.PI.Null.bGH (AAV8-TBG-NULL) (Addgene, 107787-AAV8, 105536-AAV8, respectively) were diluted in sterile saline and administered via tail vein injection to 8-10 week old male Egfr^{Fl/Fl} mice. Experiments were begun 1 week following viral particle injection, and successful deletion was confirmed at the termination of the experiment. All experiments involving mice were performed under protocols AC-AAAQ8474 and AC-AABD8554 approved by the Columbia University Irving Medical Center (CUIMC) Institutional Animal Care and Use Committee. All mouse strains were maintained in the CUIMC animal facility in accordance with institutional guidelines.

### Example 2

### Mouse Liver Injury Models

For induction of CC14-mediated fibrosis, 8-10-week-old male mice were treated twice weekly with 0.5 μL/g CC14 (Sigma, Cat. #319961) for 9 weeks. CC14 was diluted 4-fold in corn oil (Sigma, Cat. #C8267) prior to intraperitoneal injection. For NASH-mediated liver injury, 10-12-week-old male mice were fed the FPC diet (Envigo, TD.190142) for 16 weeks, and the drinking water was supplemented with 23.1 g/L fructose (Sigma, Cat. #F2543) and 18.9 g/L glucose (Sigma, Cat. #49159), or 10-12-week-old male mice were fed the choline-deficient, amino acid supplemented, high fat diet (CDAA-HFD) for 8 weeks (Research Diets, Cat. #A06071302). For insulin resistance without NASH, male mice were fed the high fat diet (HFD, 60% kcal from fat, TestDiet, Cat. #58Y1) for 8 weeks. At the end of each experiment, mice were euthanized, and the livers were removed for further processing. In diet experiments, mice were fasted for 6 hours prior to euthanasia. In some experiments, blood was collected via cardiac puncture for serum analysis. For fibrosis quantification, matched liver lobes were fixed in 10% formalin, paraffin-embedded, and stained with Sirius Red. For Sirius Red staining quantification, 6-10 random images (10× magnification) were taken of each liver section and quantification of Sirius Red positive area was performed using ImageJ. Serum ALT levels were measured on a Heska Element HT5 as per the manufacturer's protocol. Liver triglycerides were extracted using the Folch Extraction method. Briefly, liver tissue was homogenized in PBS. A chloroform methanol mixture (12 mL) was added to the homogenate (3 mL) to create an 8:4:3 ratio, the mixture was centrifuged, and the organic layer was isolated. The remaining mixture was again extracted, with addition of 10 mL solution of chloroform, methanol, and water (86:14:1). Following centrifugation, the organic layer was again isolated. The combined organic layer extract was then dried via nitrogen gas two times, the second following addition of 15% Triton X-100 in chloroform. The lipids were reconstituted in water and quantified using the Infinity triglyceride reagent (Thermo Scientific), as per the manufacturer's protocol. Serum insulin levels were measured via ELISA (Mercodia) as per the manufacturer's protocol. Metabolic testing was performed after 16 weeks on the FPC diet or 8 weeks on the HFD. For glucose and pyruvate tolerance tests, mice were fasted for 16 hours, then administered 1.5-2 g/kg glucose or 2 g/kg pyruvate via intraperitoneal injection. Blood glucose was measured prior to injection, and at 15-, 30-, 60-, 90-, and 120 minutes post-injection. For IL-6/IL-6R signaling blockade prior to the glucose tolerance testing of mice fed FPC diet, 48 hours prior to fasting, an isotope control (Bio X Cell, Clone LTF-2, 10 mg/kg) or anti-mouse IL-6R antibody (Bio X Cell, Clone 15A7, 10 mg/kg) was injected intraperitoneally. For insulin tolerance tests, mice were fasted 6 hours, then injected with 0.75 U/kg insulin (Sigma, Cat. #11376497001) via intraperitoneal injection. Blood glucose was measured prior to insulin injection, and 20-, 40-, 60-, 90-, and 120 minutes post-insulin injection.

### Example 3

### Immune Cell Isolations

Splenic cell suspensions were prepared and red blood cells were lysed in ammonium chloride (ACK) buffer. For some experiments, CD4⁺ T cells were then enriched using the DYNABEADS® FlowComp mouse CD4 kit (Life Technologies) prior to staining. For isolation of liver leukocytes, the portal vein was cut, and mice were perfused with ice cold PBS. The liver was then minced and digested in RPMI1640 supplemented with 5% fetal bovine serum (FBS, Corning), HEPES, GlutaMAX and Pen/Strep, with collagenase A (1 mg/mL, Sigma) and DNAse I (0.5 μg/mL, Sigma) at 37° C. for 45-60 minutes with constant shaking. Following a wash in RPMI1640, lymphocytes were enriched via Percoll (GE Healthcare) density centrifugation (44% layered over 67%). After centrifugation, the lymphocyte layer was collected and washed, and following lysis of red blood cells with ACK buffer, cells were stained (see below). For flow cytometric analysis of myeloid cells, cells were stained directly following digestion without Percoll enrichment. For analysis of VAT lymphocytes, VAT was minced and digested in DMEM supplemented with 2% FBS and 1.5 mg/mL Collagenase type II (Sigma) for 25 minutes. Red blood cells were then lysed with ACK buffer, and cells were then stained.

### Example 4

### Flow Cytometry Analysis

For flow cytometry analysis, cells were labeled with Ghost Dye cell viability reagent (Tonbo Biosciences). The following antibodies were used for cell surface staining: anti-CD45, -Ly-6C (BioLegend), -CD19, -CD8a, -F4/80, -Ly-6G, -CD11b, -CD11c (Tonbo Biosciences), -CD3e, -TCRs, -CD4, -NK1.1, -MHCII (BD Biosciences). The following antibodies were used for intracellular staining in conjunction with the Foxp3/transcription factor staining buffer set (Tonbo Biosciences) as per the manufacturer's protocol: anti-Foxp3 (eBioscience), -AREG (R&D). Secondary staining was performed following primary intracellular staining of Areg using strepdavidin-BV421 (BioLegend). Samples were acquired on an LSR Fortessa Flow Cytometer (BD Biosciences) and data were analyzed using FlowJo software (BD Biosciences).

### Example 5

### Immunofluorescence Microscopy Studies

To induce liver damage, hepatic stellate cell lineage trace mice (Rosa26RtdTomatoLrat-Cre) were either fed the FPC diet (as above) for 16 weeks or the CDAA-HFD for 8 weeks or injected with CC14 twice weekly for 3-4 weeks (as above). Liver lobes were fixed in 4% paraformaldehyde (Electron Microscopy Sciences) in PBS for 1 hour at room temperature, followed by dehydration in 30% sucrose in PBS overnight at 4° C. Liver samples were snap frozen in optimized cutting temperature compound (Fisher Healthcare), and 10 μm sections were cut and fixed for 10 minutes with ice cold acetone. For cleaved caspase-3 staining, liver sections were stained with anti-Cleaved Caspase-3 (Asp175) monoclonal antibody (Cell Signaling) as per the manufacturer's protocol. For Treg localization studies, after antigen retrieval (Antigen Unmasking Solution, Tris-Based, Vector Laboratories), slides were blocked with horse serum (Gibco) in PBS+0.3% Triton X-100. Primary staining with rabbit anti-CD4 (Abcam, ab183685) and rat anti-Foxp3 (Invitrogen, 14577382) was performed overnight in PBS+0.3% Triton-X100 with 1% horse serum at 4° C.

Slides were washed in PBS+0.3% Triton-X100, followed by secondary staining with donkey anti-rat AF488 and anti-rabbit Cy5 (Jackson Immunoresearch) in PBS+0.3% TritonX100 with 1% horse serum. Tile scan images were acquired on a Nikon Ti Eclipse confocal microscope using 20× magnification. Quantification of the distance between Foxp3⁺ and CD4⁺ cells to tdTomato⁺ cells was performed using a nearest neighbor search algorithm in R.

### Example 6

### Hepatic Stellate Cell Isolation and Culture in Vitro

Quiescent hepatic stellate cells were isolated as described previously, with 200,000 cells plated per well in a 24-well plate in Medium 199 (Gibco; Cat. #11150059) supplemented with PenStrep and 10% FBS. For in vitro conditioned media hepatocyte glucose production assays, culture media was also placed in cell-free wells. After 5 hours, the cell culture media was changed to 0.1% FBS in Medium 199 with PenStrep and recombinant murine Areg (R&D Systems; 500 ng/mL) was added. For hepatocyte glucose production assays, conditioned media was harvested 23 hours after Areg-treatment began. For analysis of gene expression changes, after 4 hours, RNA was extracted using TRIZOL® Reagent (Invitrogen). For gene expression analysis by RT-qPCR, cDNA was synthesized 652 using qScript cDNA SuperMix (QuantaBio) and qPCR was performed using Maxima SYBR Green qPCR Master Mix (Thermo Scientific). Ct values were normalized to 18 s levels. RNA-Seq of hepatic stellate cells was performed by the Columbia Genome Center as follows: a poly-A pull-down was performed to enrich mRNAs from total RNA samples, followed by library construction using Illumina TruSeq chemistry. Libraries were then sequenced using an Illumina NovaSeq 6000 at the Columbia Genome Center. RTA (Illumina) was used for base calling and bcl2fastq2 (version 2.19) was used for converting BCL to fastq format, coupled with adaptor trimming. Pseudoalignment was performed to a kallisto index created from transcriptomes (Ensembl v96; Mouse: GRCm38.p6) using kallisto (0.44.0).

### Example 7

### Hepatic Stellate Cell Single Cell RNA-Seq in NASH

For hepatic stellate cells single cell RNA-sequencing, male Areg^{Fl/Fl} or AregFl/FIFoxp3YFP-cre mice were fed the CDAA-HFD as above for 8 weeks. Hepatic stellate cells were enriched as described previously, then viable nucleated cells were sorted using a FACS Aria cell sorter (BD Biosciences). At the Columbia Genome Center, libraries were prepared using the 10× Genomics Single Cell Gene Expression 3′ workflow and sequencing was performed on an Illumina NovaSeq 6000. CellRanger 6.1.2 was used to process the sequencing data. Analysis was subsequently performed using Seurat 4.2.0, using standard commands. Network analysis approaches were also used to analyze transcription factor activity in the hepatic stellate cell clusters of the single cell RNA-Seq. ARACNe-AP was used to identify the transcription factor regulon utilizing bulk RNA Seq of hepatic stellate cells as described previously (100 bootstraps; p value threshold of 10-8). msVIPER was then used to infer the relative transcription factor activity in each sample, as described previously. The fibrosis activity score for each transcription factor was generated by calculating the sum of the product of the TF mode (direction of the interaction; −1 to 1) and likelihood (strength of the interaction; 0 to 1) outputs of the ARACNe-AP analysis for a set of pro-fibrotic genes (Col1al, Col1a2, Col3a1, Col5a1, Acta2, Timp1, Lox).

### Example 8

### In Vitro Metabolic Assays

The primary hepatocyte glucose production assay was adapted from previous studies. To isolate primary hepatocytes, the liver was digested in situ via retrograde perfusion of 20 mL of EGTA-based solution followed by 50 mL enzyme buffer solution containing collagenase IV (Worthington, LS004188; 0.4 mg/mL). The liver was removed and dissociated to a single cell suspension in HBSS. The single cell suspension was washed twice with HBSS, and dead cells were removed via 35% Percoll density gradient centrifugation. Hepatocytes were resuspended in Medium 199 with Pen/Strep and 10% FBS and plated.

To generate collagen-coated plates, collagen (Corning, 354236) was diluted in water for the indicated densities and plated for 1 hour at 37° C. Wells were washed with PBS prior to plating hepatocytes. For collagen density and recombinant protein assays, 100,000 cells were plated per well in a collagen-coated (for recombinant protein assays, 12.6 μg/cm²) 24-well plate.

For hepatic stellate cell conditioned media experiments, 50,000 hepatocytes were plated per well in a collagen-coated (12.6 μg/cm²) 48 well plate. Two hours after plating hepatocytes, the seeding medium was replaced. Four hours later, the hepatocytes were placed in starving medium overnight: Medium 199 with Pen/Strep for collagen density experiments, Medium 199 with PenStrep supplemented with recombinant murine IL-6 (R&D systems; 406-ML; 10 ng/mL), CTGF (R&D systems; 9190-CC; 50 ng/mL), IGFBP-1 (R&D systems; 1588-B1-025; 100 ng/mL), STC-1 (R&D systems; 9400-SO; 50 ng/mL), osteopontin (R&D systems; 441-OP; 10 ng/mL), or hepatic stellate cell-conditioned media as described above for conditioned media experiments.

To neutralize the effect of the supplemented recombinant Areg on hepatocytes, Areg neutralizing antibody (R&D Systems; AF989; 1 μg/mL) was added to conditioned media prior to hepatocyte culture. 24 hours after initial plating, hepatocytes were washed twice with PBS and glucose production medium was added to each well. Glucose production medium consisted of phenol red-free, glucose-free DMEM (ThermoFisher, A1443001) supplemented with 10 mM HEPES, 2 mM GlutaMAX, 2 mM sodium pyruvate (Sigma, P2256) and 20 mM sodium lactate (Sigma, L7022). To assess glucose production, pCPT-CAMP (Sigma, C3912; 100 μM) and dexamethasone (ThermoFisher, A13449; 1 μM) were added to glucose production media. Following a 6-hour incubation, glucose content in the supernatant was determined by a glucose-oxidase/peroxidase reaction (Sigma, GAGO20).

### Example 9

### Treg Cell RNA-Sequencing and Analysis

Foxp3GFP male mice were intraperitoneally injected twice weekly for 4 weeks with CC14 or fed the CDAA-HFD for 8 weeks, then liver, spleen and/or VAT cell suspensions were prepared and stained as described above. For bulk RNA-Seq analysis, Treg (CD4+GFP+) and Tconv (CD4⁺GFP⁻) cells were sorted from liver and spleen samples on a FACS Aria cell sorter (BD Biosciences) into TRIZOL® Reagent (Invitrogen). RNA-sequencing was performed at the Columbia Genome Center. Clontech Ultra Low v4 kit was used for cDNA synthesis followed by Nextera XT library preparation, followed by sequencing using an Illumina NovaSeq 6000. RTA (Illumina) was used for base calling and bcl2fastq2 (version 2.19) for converting BCL to fastq format, coupled with adaptor trimming. Pseudoalignment was performed to a kallisto index created from transcriptomes (Ensembl v96, Mouse: GRCm38.p6) using kallisto (0.44.0). Differential gene expression analyses were performed using the DESeq2 package. Human single cell RNA-sequencing data were previously generated and were accessed from Gene Expression Omnibus (GEO) accession GSE136103.

For single cell RNA-Seq, liver, VAT and spleen cells from Foxp3GFP mice fed normal chow or the CDAA-HFD for 8 weeks were prepared as above. Prior to fluorescent antibody staining, cells from each mouse were stained with a distinct TotalSeq-C hashtag antibody (BioLegend), as per the manufacturer's protocol. Treg cells were then sorted as above (CD4⁺GFP⁺). At the Columbia Genome Center, libraries were prepared using the 10× Genomics Single Cell 5′ workflow for gene expression, cell hashing, and full-length V (D) J T cell receptor sequencing. Sequencing was performed on an Illumina NovaSeq 6000 and CellRanger 6.1.2 was used to process the sequencing data. Analysis was subsequently performed using Seurat 4.2.0, using standard commands. Network analysis approaches were also used to analyze transcription factor activity in the Treg cell clusters of the single cell RNA-Seq. ARACNe-AP was used to identify the transcription factor regulon utilizing bulk RNA-Seq of Treg cells as described previously (100 bootstraps; p value threshold of 10-8) 78. msVIPER was then used to infer the relative transcription factor activity in each population of interest, as described previously.

### Example 10

### Human Liver Staining

Formalin-fixed, paraffin embedded (FFPE) liver samples were obtained from core needle biopsies NASH patients) or non-cancerous tissue from surgical resections (normal liver). Consecutive 3 μm thickness sections from FFPE tumor tissues were cut and transferred onto positively charged glass slides (Superfrost Plus). Slides were dried overnight and stored at 4° C. until use. The Ventana Discovery Ultra staining platform was used. FOXP3 (clone 744 SP97, Abcam, 1:50 dilution) and aSMA (clone 1A4, Dako, 1:200 dilution) were double stained using the DISCOVERY ChromoMAP DAB kit (Roche) and DISCOVERY Yellow kit (Roche), respectively, as per the manufacturer's protocol. CD3 (polyclonal, Dako, 1:100 dilution) and CD8 (clone SP16, Abcam, 1:100 dilution) were double stained using the DISCOVERY ChromoMAP DAB kit and DISCOVERY Purple kit (Roche), respectively, as per the manufacturer's protocol. Cell counting of FOXP3⁺ and CD3⁺ cells was performed manually by a trained pathologist blinded from clinical data and results were expressed as a ratio of number of cells per 10 mm² of liver tissue. The whole tissue area was considered for biopsies and a representative area was selected for the surgical specimens.

### Example 11

### Statistical Analysis

Statistical analyses were performed in GraphPad Prism 9. Two-sided unpaired t-tests or 2-way ANOVA with post-hoc Holm-Sidak tests were used, as applicable. p<0.05 was considered statistically significant.

### Example 12

### Areg Production by Treg Cells Promotes Liver Fibrosis

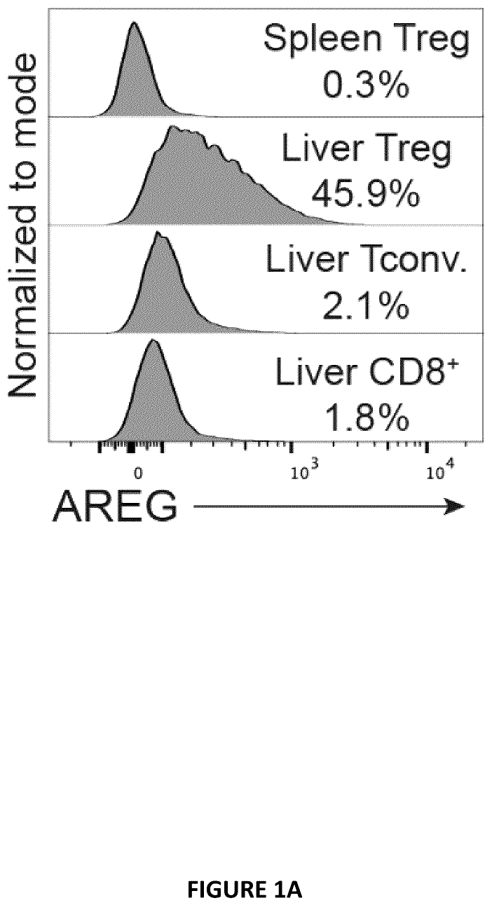

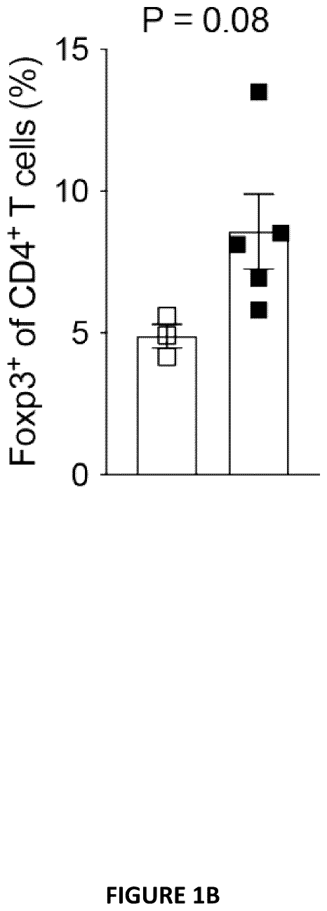

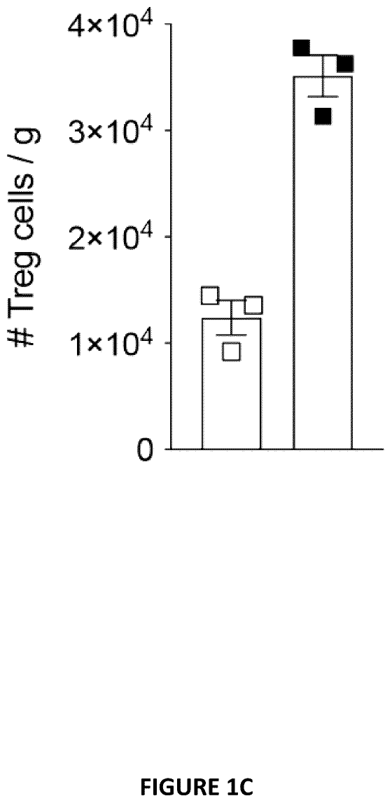

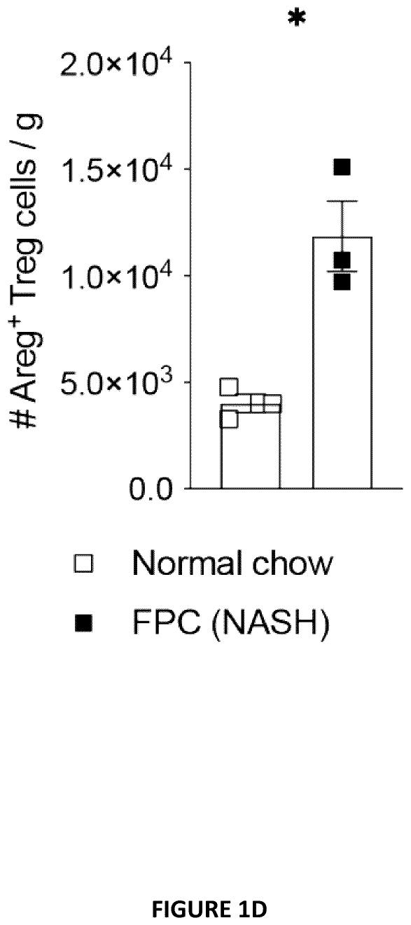

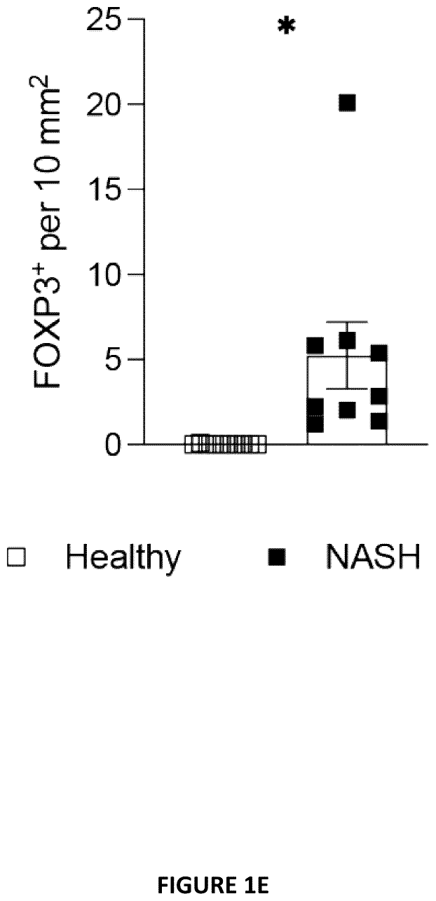

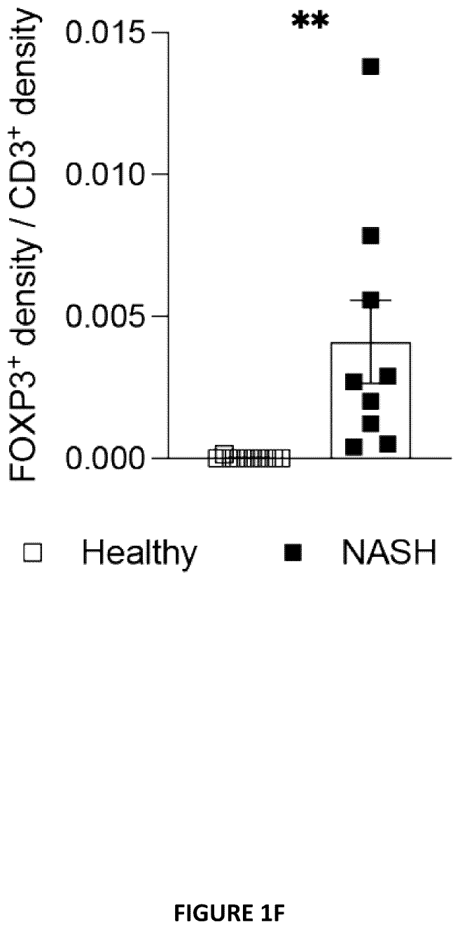

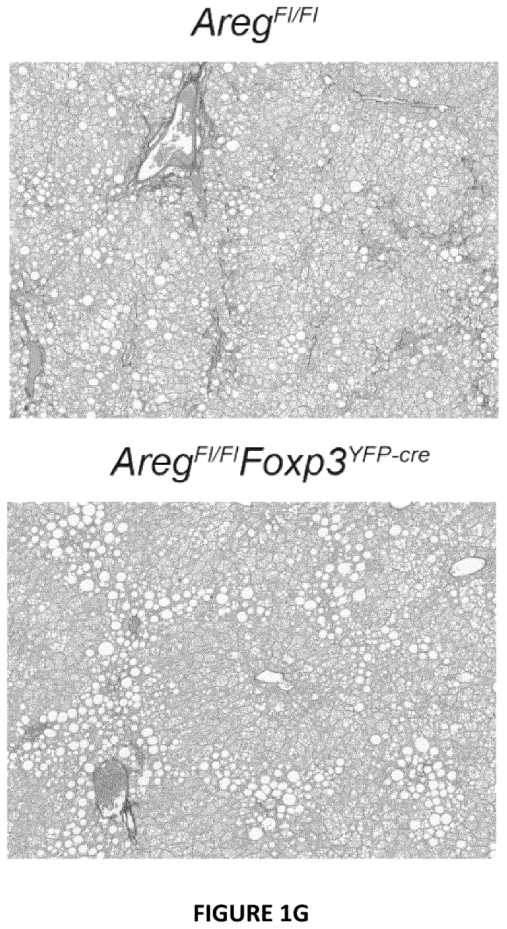

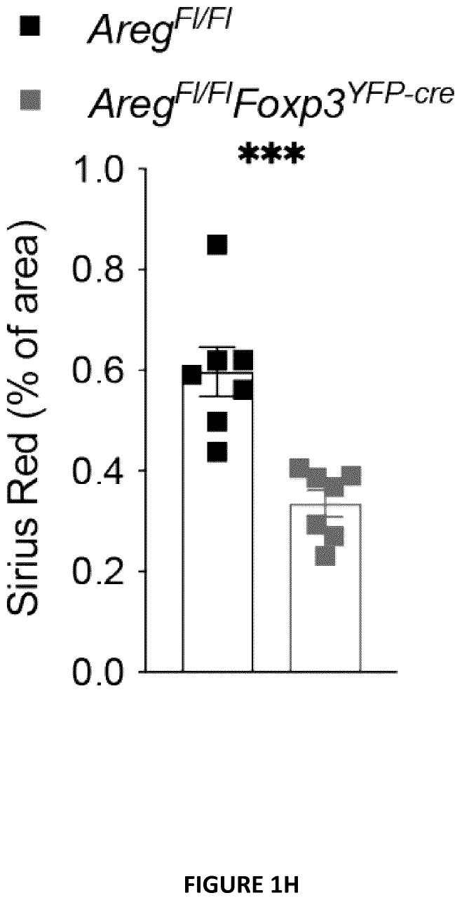

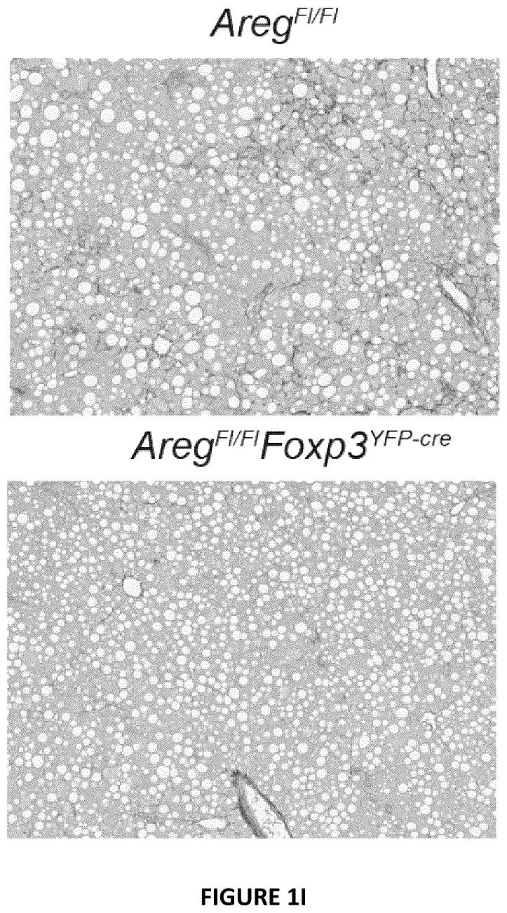

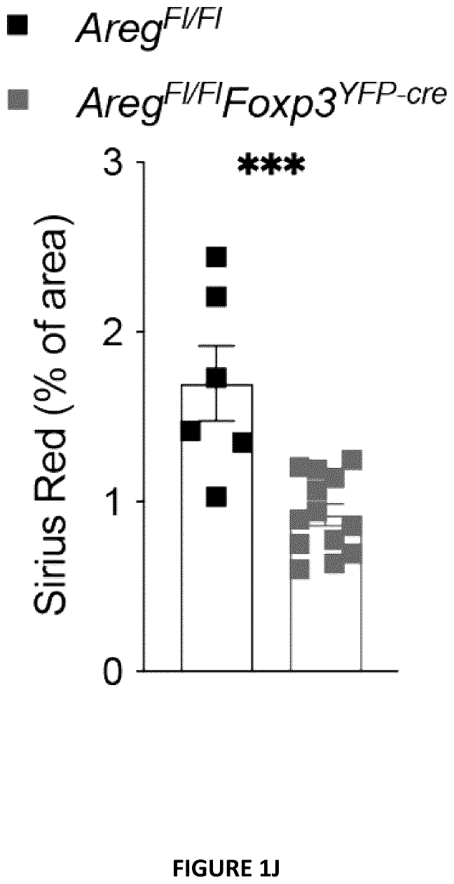

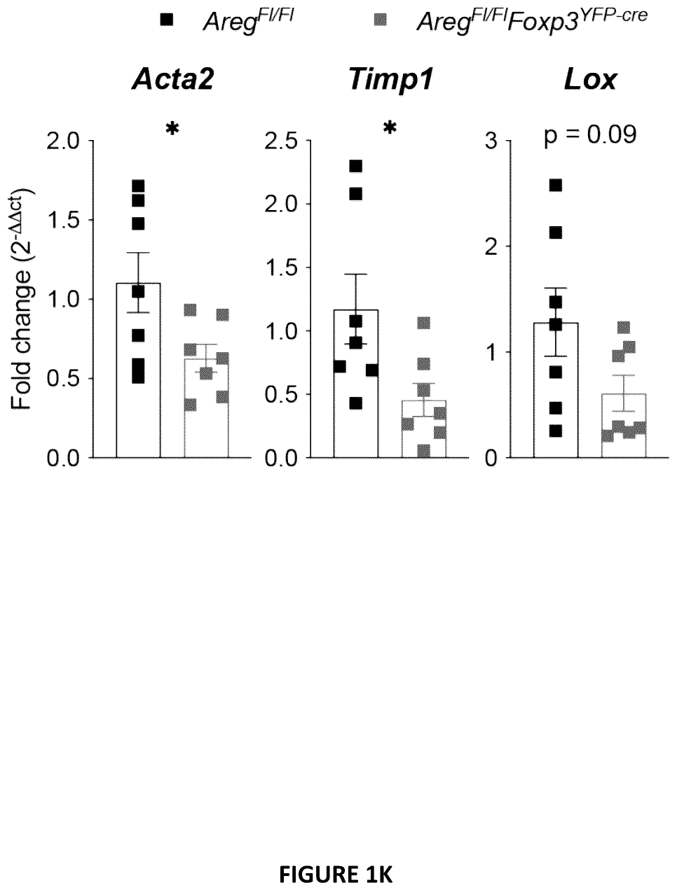

To study the tissue reparative role of Treg cells in the liver, the present inventors first analyzed liver T cell subsets for their expression of Areg, focusing on Areg as a potential Treg cell-derived mediator that is critical to tissue repair. In healthy wildtype mice, liver Treg cells produce substantial amounts of Areg protein relative to spleen Treg cells and, most notably, to other liver T cell subpopulations (FIG. 1A), suggesting that Treg cell-derived Areg may contribute to liver homeostasis. Consistent with previous work, following chronic carbon tetrachloride (CC14)-induced injury—a toxin based model that leads to robust liver fibrosis and in which germline deletion of Areg has been shown to be protective—increased frequencies of liver Treg cells were detected (data not shown). In addition, the present inventors observed significant increases in both the overall Treg cell number and, more specifically, the number of Areg-producing liver Treg cells (data not shown). To investigate the role of Areg-producing Treg cells in a more clinically relevant setting, the present inventors utilized two diet induced mouse models of NASH. In mice fed the fructose, palmitate, and cholesterol rich (FPC) NASH diet for 16 weeks, which induces moderate liver fibrosis as well as insulin resistance, the present inventors observed increases in both the frequency and number of liver Treg cells when compared to animals fed normal chow (FIG. 1B-1C). Crucially, the number of Areg-producing Treg cells was also increased in NASH livers (FIG. 1D), suggesting that Treg cell-derived Areg may play a role in protection from and/or the development of NASH.

To reinforce their findings using the FPC diet, the present inventors utilized the choline-deficient, L-amino acid-defined high fat diet (CDAA-HFD) model of NASH—which induces substantial fibrosis (data not shown)—and similarly observed increased frequencies and numbers of liver Treg cells and greater total numbers of Areg-producing Treg cells (data not shown). the present inventors also examined whether Treg cells were increased in NAFLD without NASH. Notably, liver Treg cells were not enriched in the widely used high fat diet (HFD) that induces robust steatosis but fails to progress to NASH (data not shown). Additionally, liver Treg cells were not enriched at an earlier timepoint (8 weeks) on the FPC diet when liver steatosis is evident, but NASH has yet to develop (data not shown), which, together, is consistent with hepatocyte injury contributing to an enrichment of Treg cells within inflamed and damaged, but not solely steatotic, livers.

To assess the clinical relevance of their findings, the present inventors examined Treg cells in human liver disease, first utilizing samples from healthy and NASH human liver tissue. Consistent with data from their murine diet-induced NASH models, the present inventors observed an increase in liver Treg cells in human NASH liver samples compared to normal liver (FIG. 1E). Further, Treg cells were enriched relative to total CD3⁺ T cells in human NASH (FIG. 1F), suggesting a disproportionate increase in Treg cells in human NASH livers, consistent with their observations in mice. Finally, the present inventors expanded their analyses to include liver specimens from different etiologies of human liver fibrosis. the present inventors observed that Treg cells were enriched in human alcoholic steatohepatitis (ASH), autoimmune hepatitis, and drug/toxin-induced liver fibrosis (data not shown), consistent with their murine findings across etiologies (data not shown) and suggesting that liver Treg cell enrichment may be common to human liver disease across major etiologies.

The present inventors next sought to functionally assess the role of Treg cell-derived Areg in liver disease and fibrosis, aiming to clarify whether Areg from Treg cells offers a protective function—as has been suggested by prior studies wherein total depletion of Treg cells was performed. Thus, the present inventors utilized Areg-conditional knockout mice crossed with lineage-specific Cre drivers to ablate Areg production from all hematopoietic cells (Vav1-Cre), myeloid cells (Lyz2^{wt/cre}), all T cells (CD4-Cre), or Treg cells (Foxp3^{YFP-cre}). First, using the chronic CC14 model, the present inventors observed a significant reduction in liver fibrosis, as measured by Sirius Red staining, in the absence of Areg derived from all hematopoietic cells (Areg^{Fl/Fl}Vav1-Cre), but not specifically from myeloid cells (Areg^{Fl/Fl}Lyz2^{wt/cre}), relative to littermate controls (Areg^{Fl/Fl}) (data not shown). Deletion of Areg in all CD4⁺ and CD8⁺ T cells (Areg^{Fl/Fl}CD4-Cre), and specifically in Treg cells (Areg^{Fl/Fl}Foxp3^{YFP-cre}), offered protection from CCl4-induced fibrosis (data not shown), consistent with a role for Treg cell-derived Areg in promoting, rather than protecting from, toxin-mediated liver fibrosis as prior Treg cell-depletion studies suggested.

To assess the role Areg in the more clinically relevant setting of NASH, Areg conditional knockout mice were fed the FPC diet. Similar to the CCl4 model, deletion of Areg from all hematopoietic cells and all CD4⁺ and CD8⁺ T cells, but not myeloid cells, inhibited the development of NASH-mediated fibrosis (data not shown). Furthermore, consistent with their observations in chronic CCl4-induced liver injury, Treg cell-derived Areg promoted the development of liver fibrosis in the context of NASH, as significantly less Sirius Red staining was observed in liver sections taken from FPC diet-fed Areg^{Fl/Fl}Foxp3^{YFP-cre} animals compared to littermate controls (FIG. 1G-1H). Mice lacking T cell-or Treg cell-derived Areg also had significantly less Sirius Red staining in liver sections than littermate controls in the CDAA-HFD model of NASH (FIG. 1I-1J), offering additional evidence that Treg cell-derived Areg promotes NASH-mediated liver fibrosis. Notably, the protection from fibrosis observed in the absence of Treg cell-derived Areg was not attributable to alterations in body weight, hepatocyte cell damage or death, liver fat accumulation, or liver inflammation, as evidenced by similar levels of weight gain, serum liver enzymes, liver triglyceride levels, and immune cell infiltration in Areg conditional knockout and littermate control mice on the FPC diet (data not shown).

Further, loss of Treg cell-derived Areg did not affect overall Areg protein levels in the livers of mice fed the FPC diet, suggesting a unique and local interaction may mediate fibrosis induction in response to Areg produced by Treg cells (data not shown). Finally, the expression of genes reflecting hepatic stellate cell activation was reduced in the livers of FPC-fed Areg^{Fl/Fl}Foxp3^{YFP-cre} mice compared to littermate controls (FIG. 1K), consistent with Treg cell-derived Areg promoting hepatic stellate cell activation and liver fibrosis. Taken together, these data demonstrate a specific expansion of liver Areg-producing Treg cells in the setting of chronic liver damage and identify, for the first time, a maladaptive role for Treg cell-derived Areg-promoting liver fibrosis following toxin induced chronic liver injury and diet-induced NASH.

### Example 13

### Treg Cells in Liver Fibrosis Exhibit a Transcriptionally Distinct Activation Profile

Taking into consideration that Treg cells in tissues—especially in settings of tissue damage and inflammation—demonstrate unique transcriptional signatures, the present inventors sought to develop a better molecular understanding of the unique features of Treg cells in the context of liver disease by RNA sequencing (RNA-Seq). To this end, the present inventors first chose to examine Treg cells from CCl4-treated mice—a model that rapidly induces robust fibrosis that is dependent on Treg cell-derived Areg (data not shown). Liver Treg cells clustered distinctly from spleen Treg cells and from liver and spleen conventional CD4⁺Foxp3⁻ T cells (Tconv), with Treg cells from fibrotic livers clustering distinctly from those at steady state (data not shown). Differential gene expression analysis revealed significant transcriptional alterations occurring specifically within liver Treg cells in the context of chronic toxin-induced injury, as compared to liver Tconv cells or splenic counterparts (data not shown), suggesting Treg cells are proficient in sensing alterations in the hepatic environment.

In support of this hypothesis, fibrotic liver Treg cells upregulate pro-inflammatory cytokine receptors and tissue-homing chemokine receptors (data not shown), representative of a unique liver-specific transcriptional program that supports their localization and activation in the setting of fibrosis. Additionally, Treg cells isolated from fibrotic versus normal livers displayed increased metabolic pathway activation, cell cycle progression, and increased cytokine receptor signaling (data not shown), consistent with an increased activation phenotype. To study human Treg cells in liver fibrosis, the present inventors examined previously published single-cell RNA sequencing data from healthy and cirrhotic human livers, specifically analyzing a cluster of Treg cells identified based on the expression of FOXP3 (data not shown). Notably, human Treg cells isolated from cirrhotic livers upregulated genes including TIGIT, CXCR4, CXCR3, IL10, CTLA4 and CCR5, and were enriched for transcriptional pathways reflecting increased activation and proliferation (data not shown), consistent with their observations for murine hepatic Treg cells isolated from fibrotic livers in the context of chronic CCl4-induced injury (data not shown).

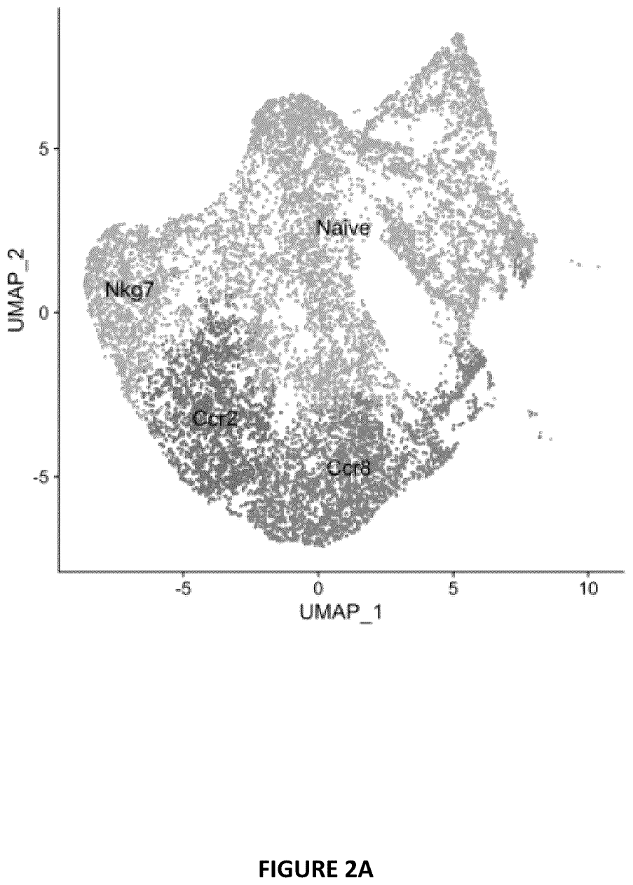

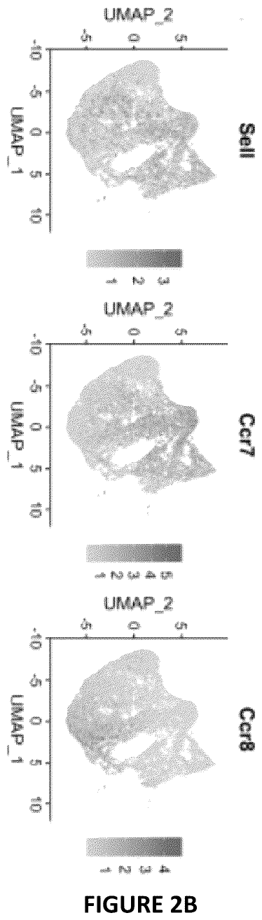

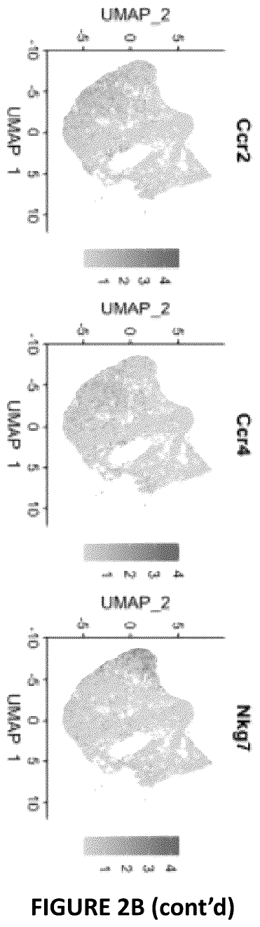

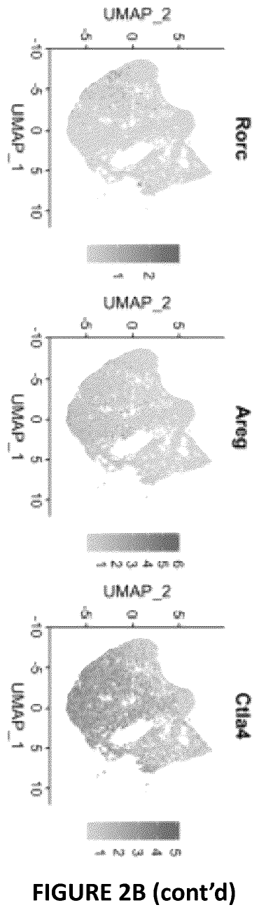

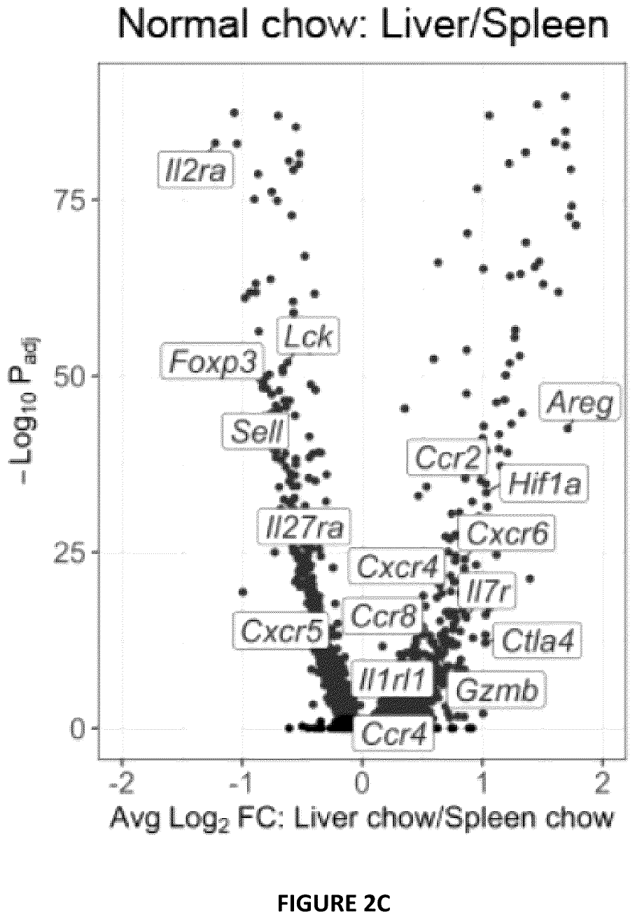

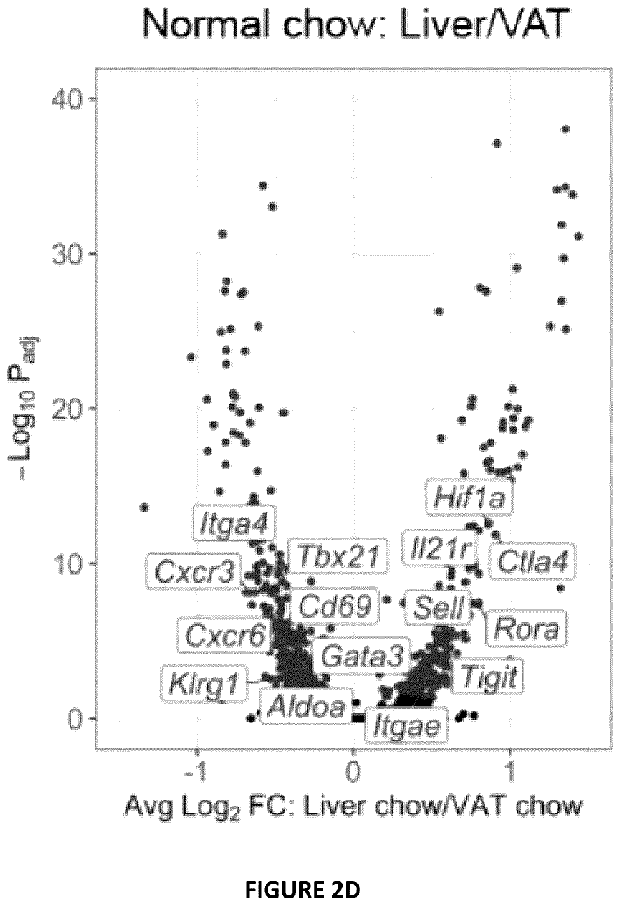

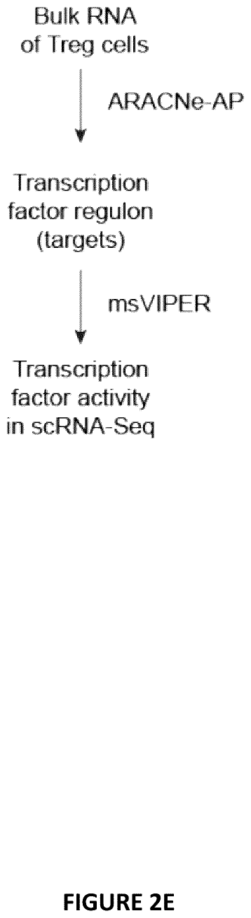

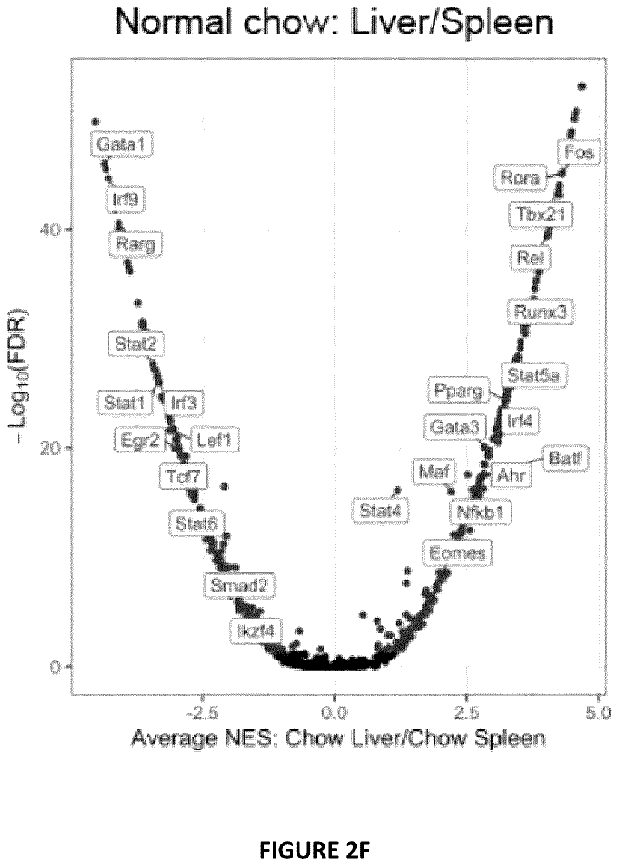

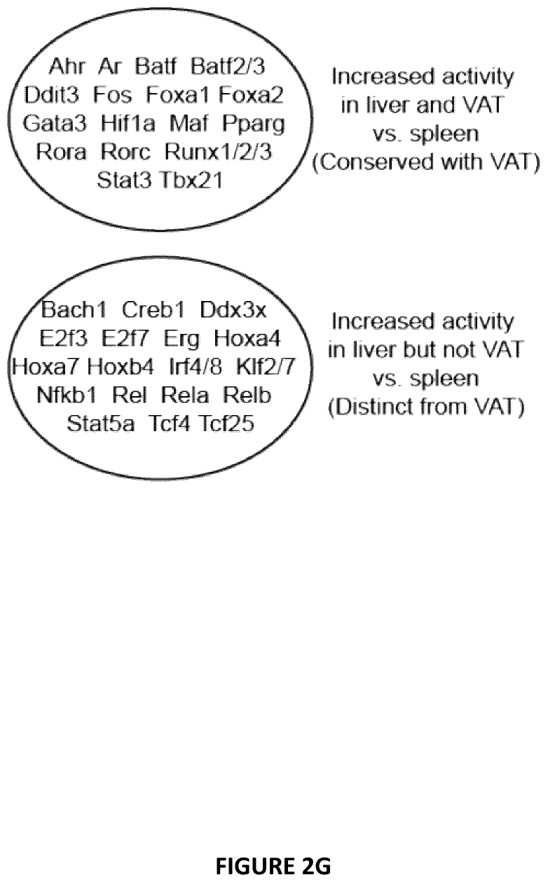

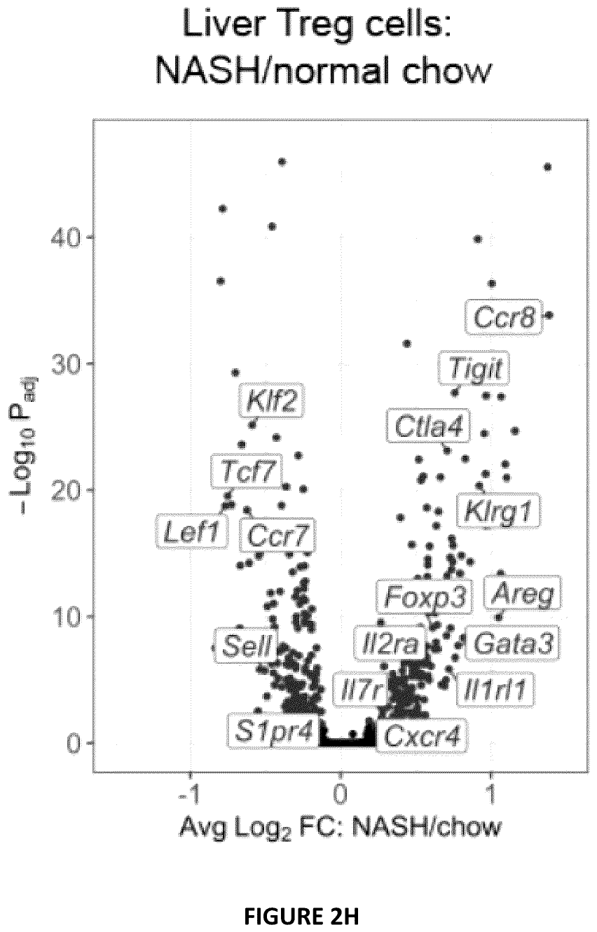

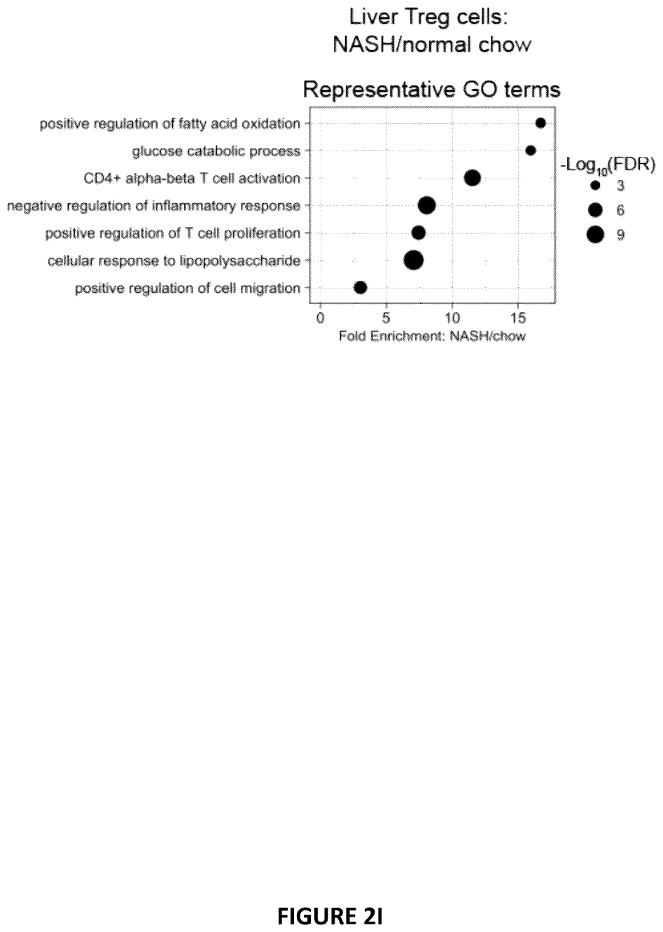

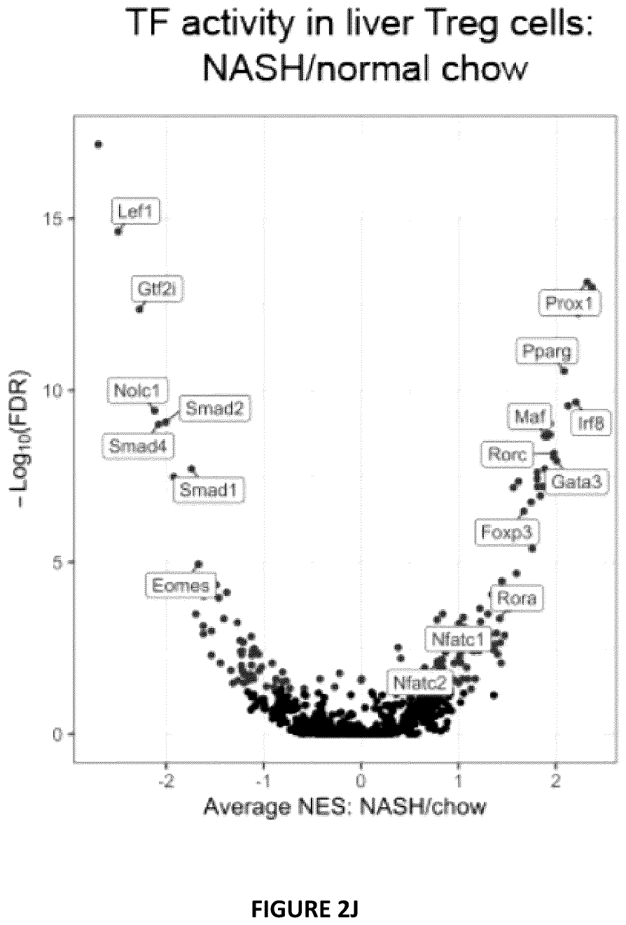

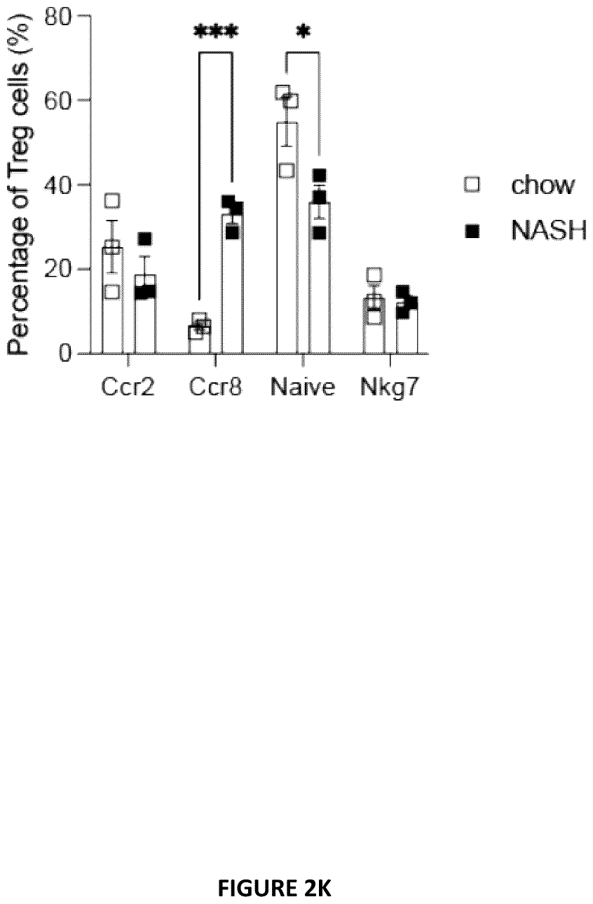

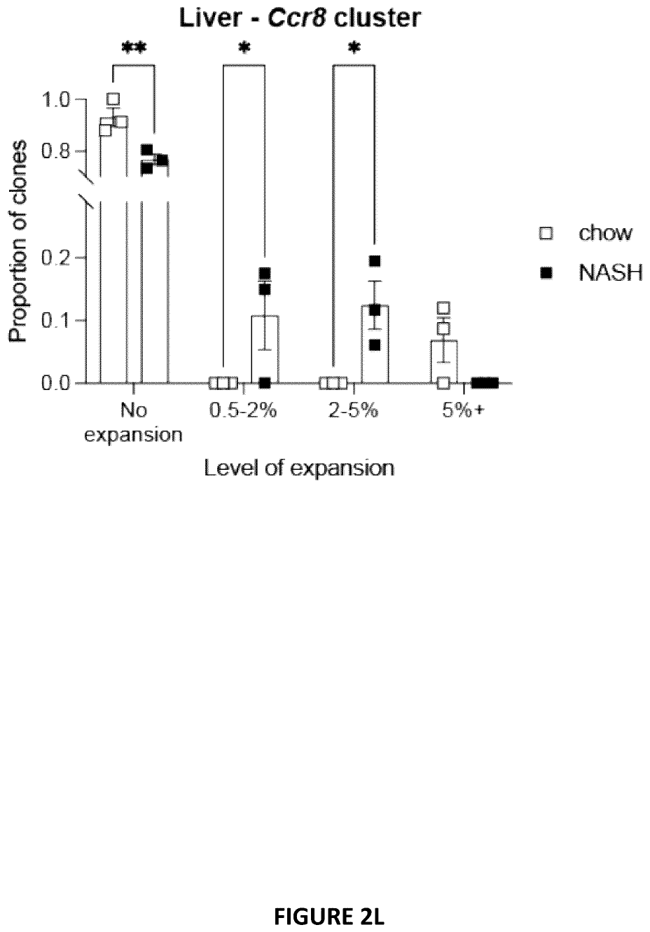

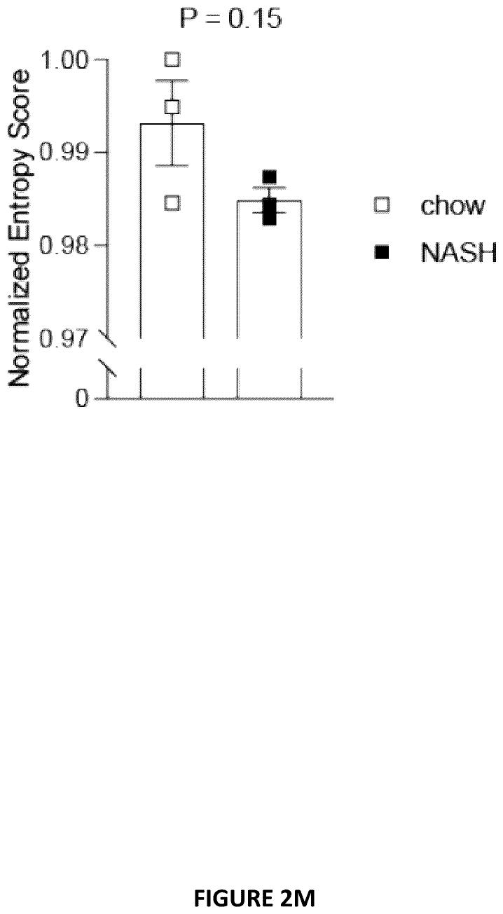

To provide a more detailed view of Treg cells at steady state and in NASH, the present inventors performed single cell RNA-Seq of Treg cells isolated from the liver, spleen, and visceral adipose tissue (VAT) of mice fed normal chow or the NASH-inducing CDAA-HFD. The present inventors chose to include VAT Treg cells in this analysis due to their documented unique transcriptome and T cell receptor (TCR) repertoire, as well as their role in protecting from metabolic disease. When considering cells from all samples, the present inventors observed four clusters of Treg cells: (1) a naive and/or circulating population characterized by expression of Ccr7 and Sell (which encodes CD62L; hereafter referred to as “naive”), (2) an activated tissue-resident cluster expressing Ccr8, (3) a memory population expressing Ccr2, Ccr4 and Rorc (hereafter referred to as the “Ccr2” cluster), and (4) a cluster expressing Nkg7 (FIG. 2A-2B). Notably, Areg was predominantly expressed by the Ccr2 and Ccr8 Treg cell clusters (FIG. 2B). At steady state, liver Treg cells were enriched for the Ccr2 cluster, while the Nkg7 population was greatest in the VAT, and the spleen was enriched for naive Treg cells (data not shown). Transcriptionally, at steady state, liver Treg cells differed significantly from spleen and VAT Treg cells, with liver Treg cells expressing distinct chemokine and cytokine receptors compared those isolated from spleen and VAT (FIG. 2C-2D). Liver Treg cells expressed higher levels of Itgae (encoding CD103) compared to VAT Treg cells, while Cd69 was more highly expressed by VAT compared to liver Treg cells (FIG. 2D), suggesting differential tissue retention molecules between the tissue Treg cell populations. Furthermore, gene ontology analysis revealed heightened responsiveness to microenvironmental cues for Treg cells isolated from liver, as compared to those from spleen or VAT (data not shown).

To identify the transcription factors responsible for the observed liver-specific transcriptional profile, the present inventors utilized the ARACNe-AP algorithm to reconstruct Treg cell transcriptional networks utilizing mutual information estimators from bulk RNA-Seq of Treg cells, which was then input into ms VIPER to score transcription factor activity in each sample (FIG. 2E). These analyses revealed that transcription factors such as T-bet, RORa, STAT5, PPARg, AHR, GATA3 and Maf had increased activity in liver Treg cells compared to spleen Treg cells at steady state, while transcription factors such as Smad2, TCF1 and LEF1 had reduced activity in liver Treg cells compared to spleen Treg cells (FIG. 2F), consistent with a transcriptional response to the liver microenvironment. Notably, this analysis revealed transcription factors whose activity were increased in both liver and VAT Treg cells compared to spleen Treg cells, including PPARg (known to regulate VAT Treg cell development), AHR, AR, HIF1a, and RORgt, as well as transcription factors that had increased activity in liver Treg cells but not VAT Treg cells compared to spleen Treg cells, such as HOXB4, IRF4, IRF8, and STAT5 (FIG. 2G). Taken together, these analyses suggest that liver Treg cells share critical transcription factors with VAT Treg cells but that a distinct set of transcription factors were also responsible for the unique liver Treg cell transcriptional program.

The present inventors then considered the effect of NASH on Treg cells. Consistent with the effect of CCl4 injury on liver Treg cells (data not shown), NASH induced substantial transcriptional changes in liver Treg cells, including significant upregulation of genes such as Il2ra, Klrg1, Areg, chemokine and cytokine receptors including Il1rl1 (encoding ST2) and Il18r1, and downregulation of genes such as the T cell stemness marker, Tcf7 (FIG. 2H). Overall, gene ontology analyses revealed an increased activation signature for Treg cells in NASH livers (FIG. 2I). The present inventors next utilized the ARACNe-AP/VIPER approach (FIG. 2E) to analyze transcription factor activity in liver Treg cells. These analyses revealed that transcription factors such as PPARg, GATA3, Maf, NFAT, and RORgt—each with known functions in Treg cells—had increased activity in liver Treg cells from NASH compared to normal chow, while SMAD family members and LEF1 had reduced activity in liver Treg cells in NASH (FIG. 2F). Taken together, these data suggest that a mixture of transcription factors drive the liver Treg cell transcriptional profile in NASH.

The present inventors next considered whether specific liver Treg cell subsets were uniquely altered. In this regard, the present inventors observed significant expansion of only the Ccr8-expressing cluster of liver Treg cells isolated from diet induced NASH mice (FIG. 2K); this cluster notably displayed gene expression changes reflective of increased activation (data not shown), suggesting Ccr8-expressing liver Treg cells are specifically activated in NASH. Finally, as TCR signaling is thought to contribute to Treg cell accumulation in the brain and VAT, the present inventors sought to examine the potential role of antigen-driven expansion of Treg cells in the setting of NASH. The present inventors observed that a greater proportion of Treg clonotypes were expanded in NASH livers than in normal chow, a pattern specific to the Ccr8-expressing cluster (FIG. 2L). Further, the present inventors noted a trend towards a reduction in the normalized Shannon entropy in this Ccr8-expressing cluster in NASH samples (FIG. 2M), suggesting reduced clonal diversity within this cluster in NASH. However, there was a noticeable lack of oligoclonal dominance as assessed by frequency of top clones in normal chow and NASH liver samples (data not shown).

Moreover, the present inventors utilized the GLIPH algorithm that is designed to identify motifs predicted to bind the same MHC/peptide antigen. Across the normal chow and NASH liver samples, the GLIPH algorithm identified 279 motifs, the vast majority of which were found in both chow and NASH TCR repertoires (data not shown), suggesting that Treg cell enrichment in NASH livers is not due to a select set of antigens and consistent with prior studies suggesting Areg production by Treg cell is independent of TCR signaling. Taken together, these data reveal that an activated subset of hepatic Treg cells isolated from fibrotic liver environments express unique transcriptional signatures reflective of their increased activation in the setting of chronic tissue damage and inflammation.

### Example 14

### Treg Cell-Derived Areg Directly Activates Hepatic Stellate Cells to Promote Liver Fibrosis

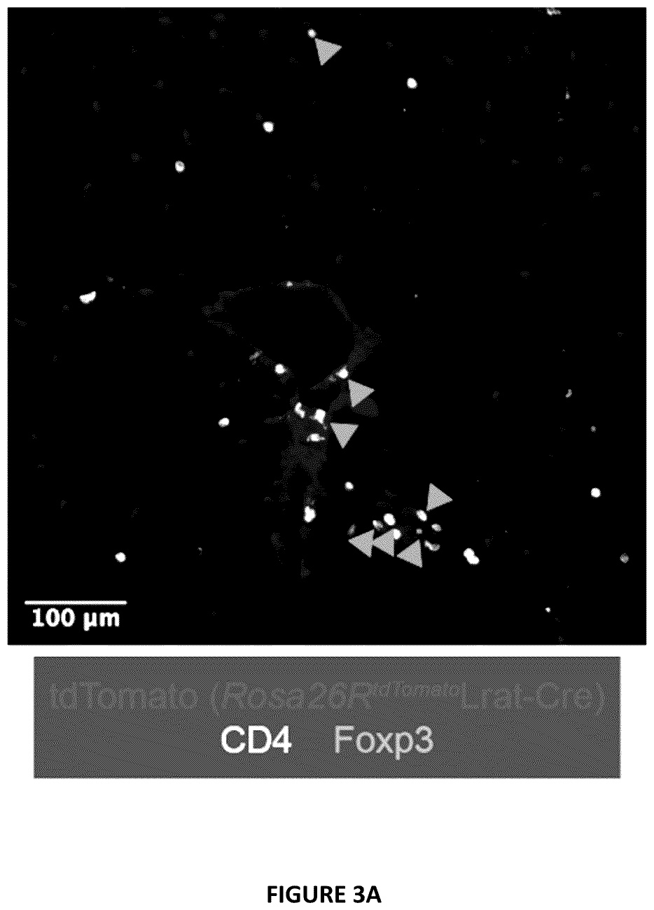

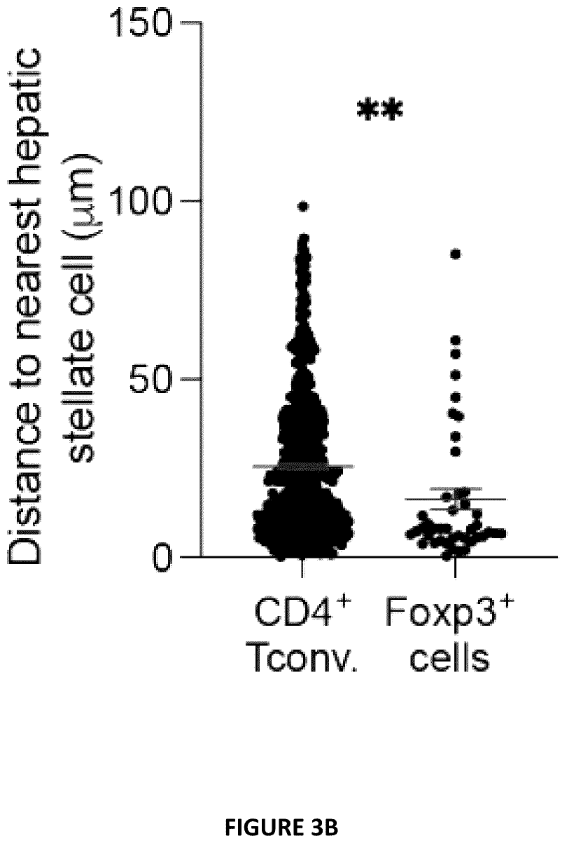

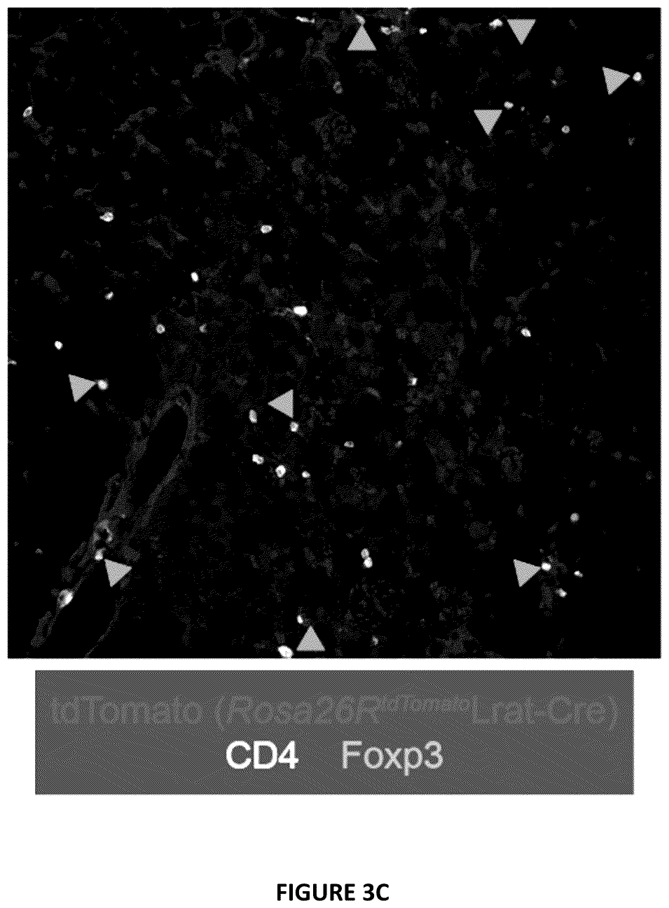

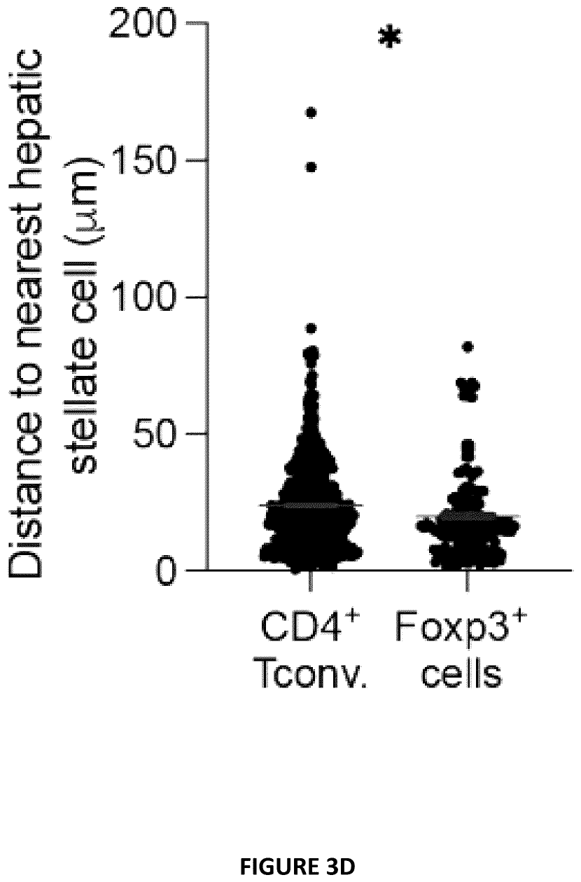

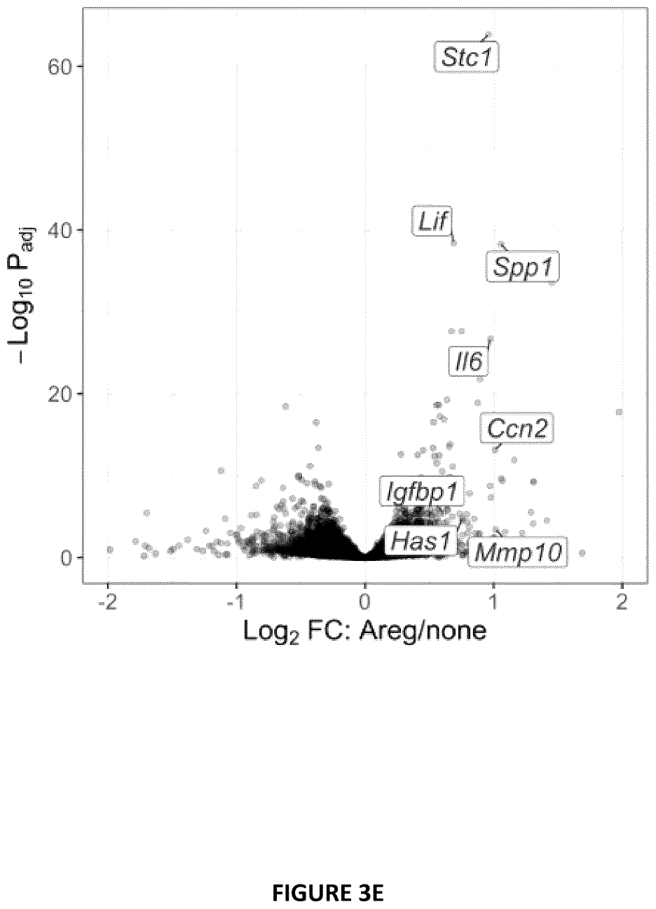

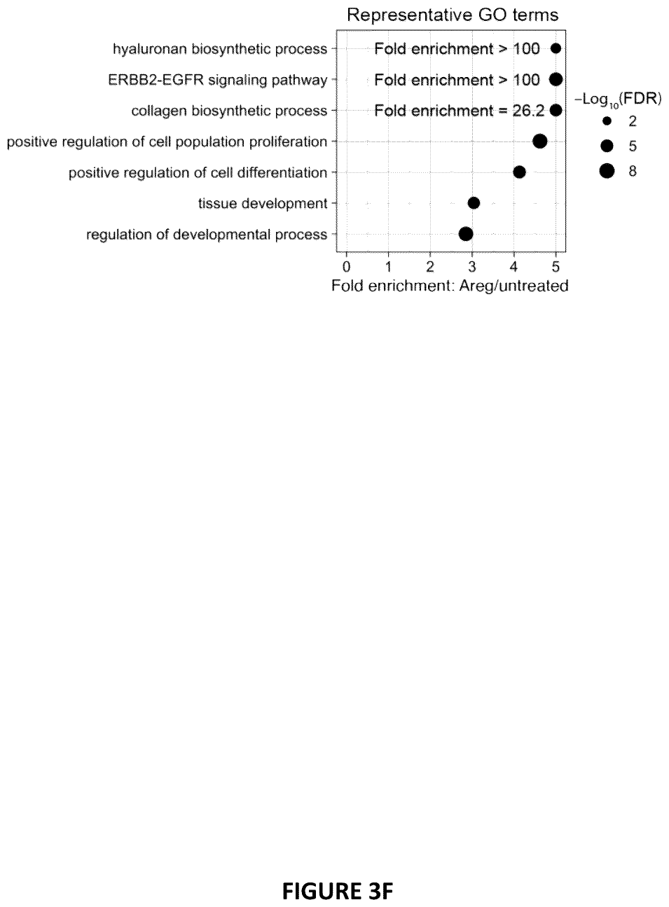

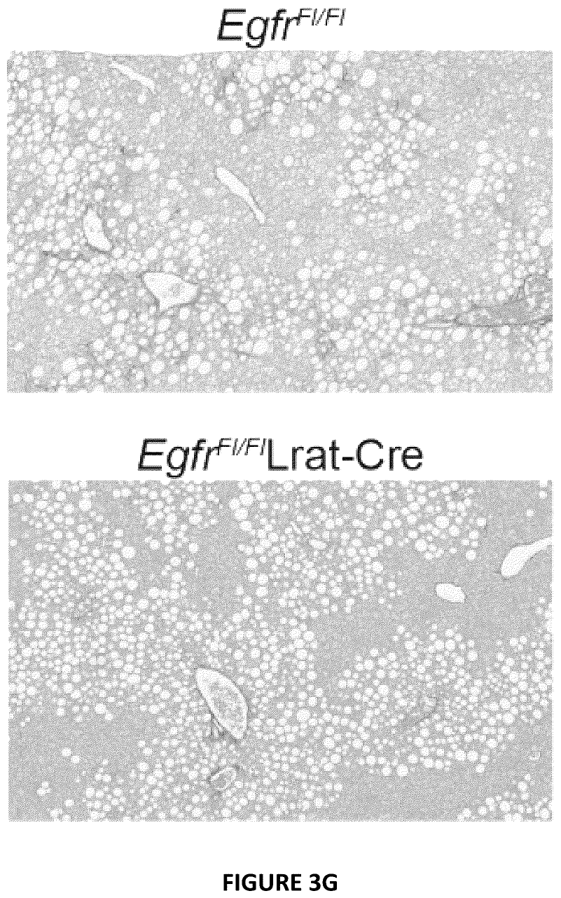

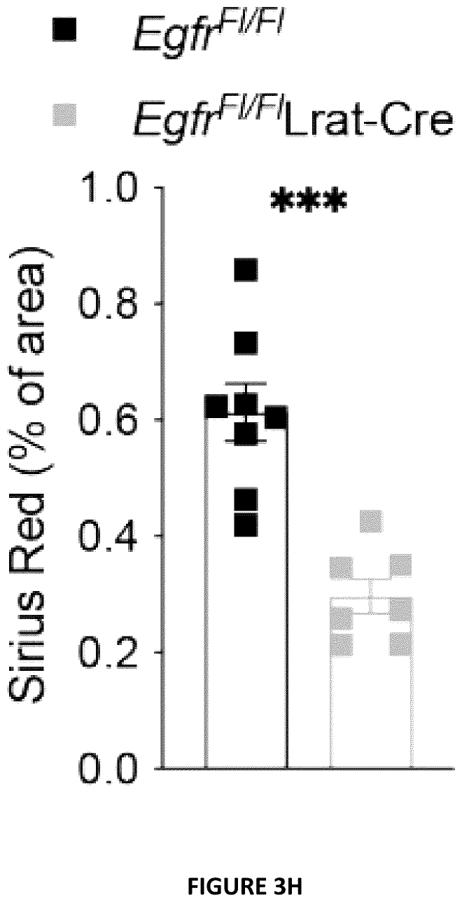

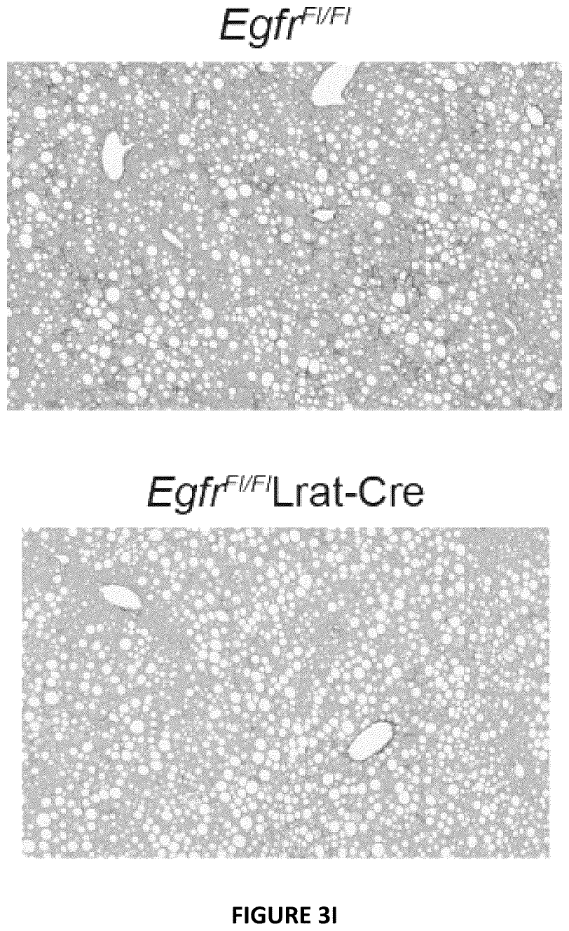

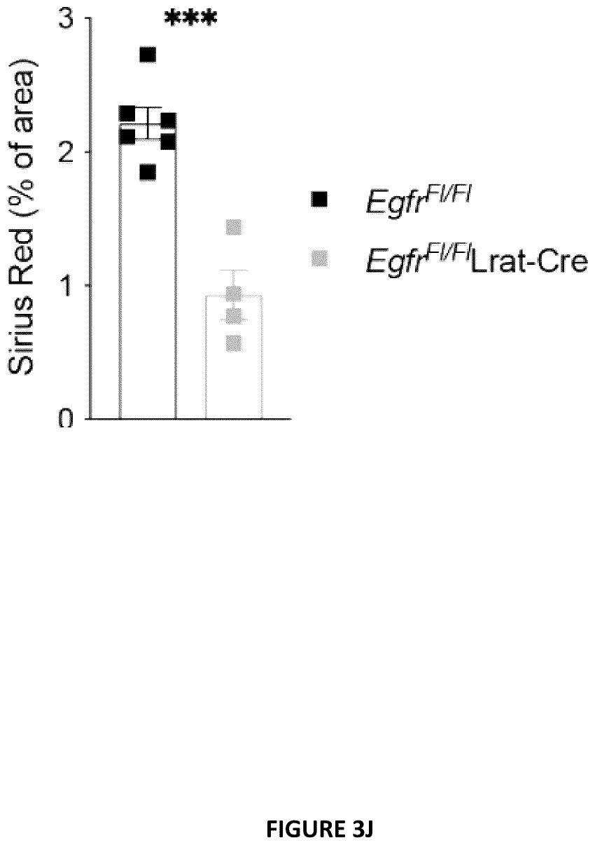

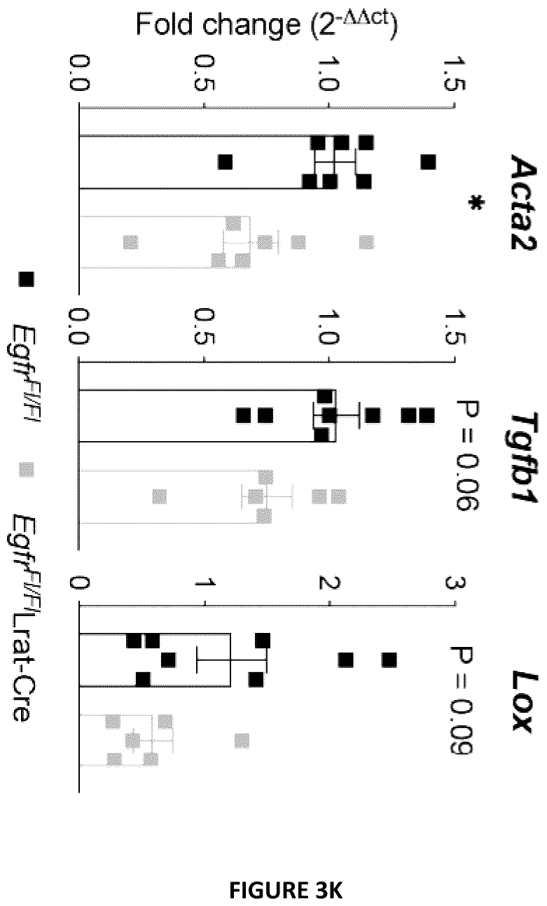

The present inventors next considered the tissue-specific cell types that may be responding to Treg cell-derived Areg to promote liver fibrosis. To this end, the present inventors investigated whether Treg cell-derived Areg directly or indirectly activates hepatic stellate cells, the predominant (>95%) source of collagen-producing cells and myofibroblasts in liver fibrosis. the present inventors hypothesized that Treg cells may localize near the specific cell type that senses Treg cell-derived Areg, reflecting a direct signaling interaction between these cells. Using a tracing approach to identify hepatic stellate cells (Rosa26R^{tdTomato}Lrat-Cre), the present inventors observed that Treg cells, but not CD4⁺Foxp3⁻ Tconv cells, colocalized with tdTomato⁺ hepatic stellate cells during chronic CCl4-induced injury (data not shown) and in the FPC and CDAA-HFD models of NASH (FIG. 3A-3D), suggesting that Treg cell-derived Areg may directly activate EGFR on hepatic stellate cells to promote liver fibrosis.

The present inventors next sought to validate these imaging findings with functional manipulations of hepatic stellate cells, first studying gene expression changes in hepatic stellate cells treated with recombinant murine AREG (rmAREG) in vitro via RNA-Seq. In response to Areg stimulation, quiescent hepatic stellate cells upregulated Lif, an EGFR signaling target gene, and genes indicative of hepatic stellate cell activation (Acta2, Timp1) (FIG. 3E). Interestingly, an increase in the expression of genes reported to be involved in hepatic insulin resistance, including Il6, Spp1 and Igfbp151 (FIG. 3E), were also observed following Areg treatment. Further, Areg induced expression of transcripts corresponding to EGFR signaling, cell proliferation and differentiation, and tissue repair and collagen biosynthesis gene expression signatures (FIG. 3F), consistent with Treg cell-derived Areg acting directly on hepatic stellate cells to promote their activation and liver fibrosis development.

To functionally study the interplay of Treg cell-derived Areg with hepatic stellate cells in vivo, the present inventors generated hepatic stellate cell-specific Egfr conditional knockout mice. Animals lacking EGFR on hepatic stellate cells (Egfr^{Fl/Fl}Lrat-Cre) exhibited no quantifiable steady state abnormalities when compared to Egfr^{Fl/Fl} littermate controls (data not shown); however, in the setting of chronic CCl4 injury (data not shown), hepatic stellate cell-specific Egfr deletion resulted in a significant reduction of liver fibrosis. Moreover, in both the FPC diet and CDAA-HFD models of NASH, mice lacking EGFR on hepatic stellate cells developed significantly less fibrosis (FIG. 3G-3J). Finally, loss of EGFR on hepatic stellate cells resulted in reduced expression of genes indicative of hepatic stellate cell activation in the livers of mice fed the FPC diet (FIG. 3K), consistent with EGFR signaling in hepatic stellate cells promoting their activation and liver fibrosis. Notably, the protection observed in these models was similar to that seen following loss of Treg cell-derived Areg (FIG. 1G-1K), offering in vivo functional evidence complementing their imaging studies (FIG. 3A-3D). Together, these findings support the hypothesis that Treg cell-derived Areg activates EGFR signaling on hepatic stellate cells to promote liver fibrosis.

### Example 15

### Treg Cell-Derived Areg Promotes the Activation of Quiescent Hepatic Stellate Cells in NASH

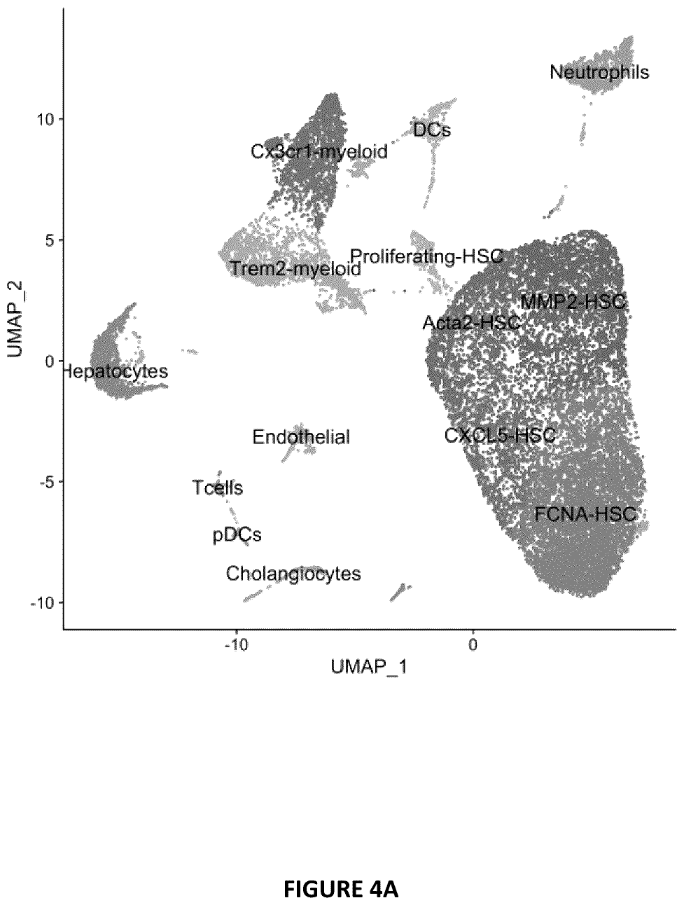

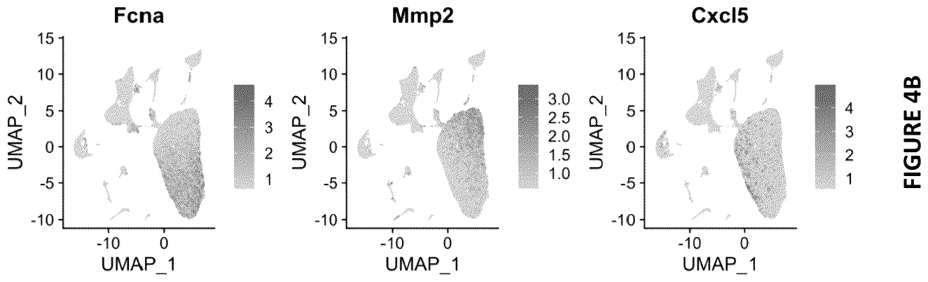

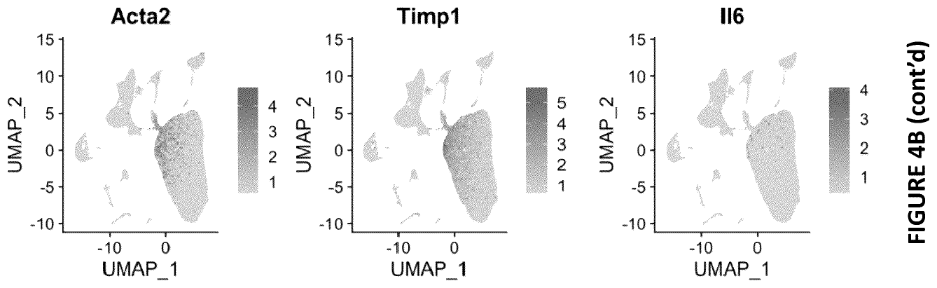

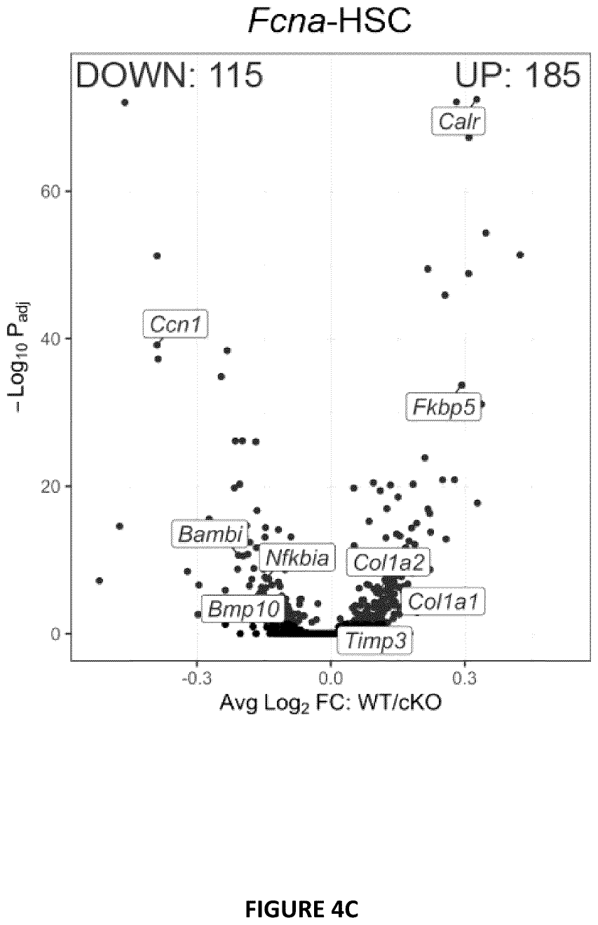

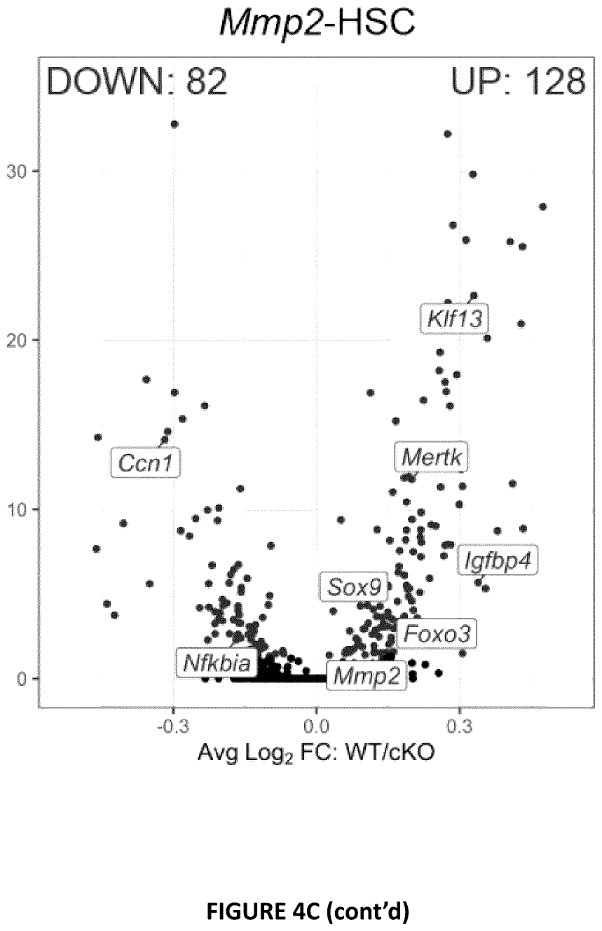

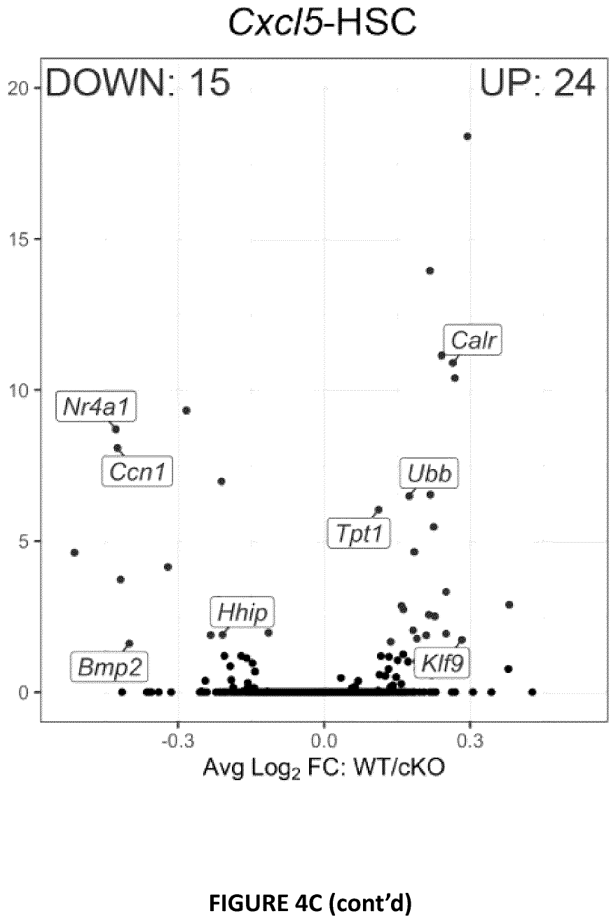

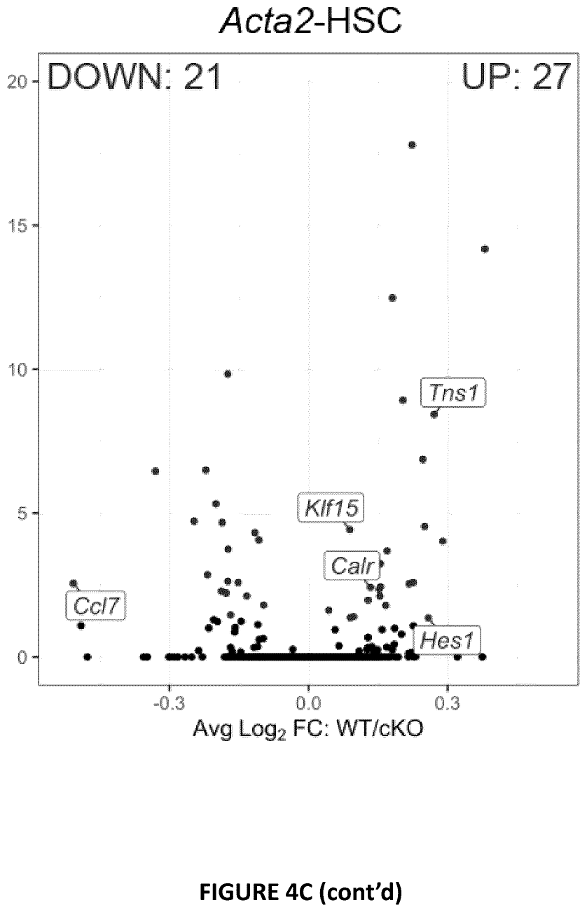

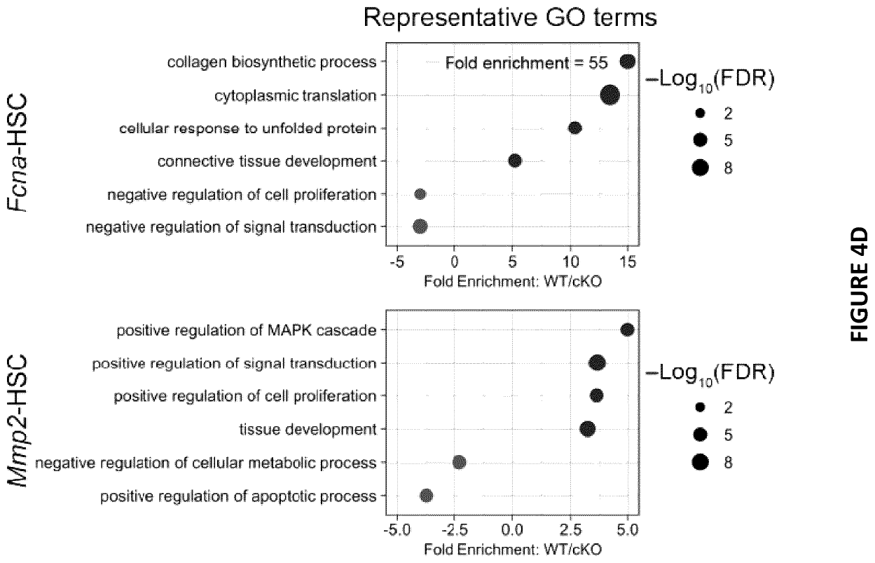

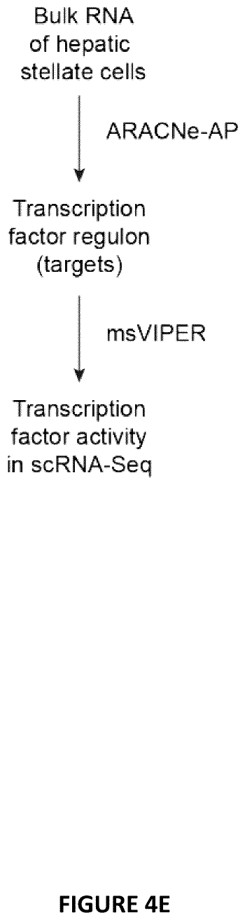

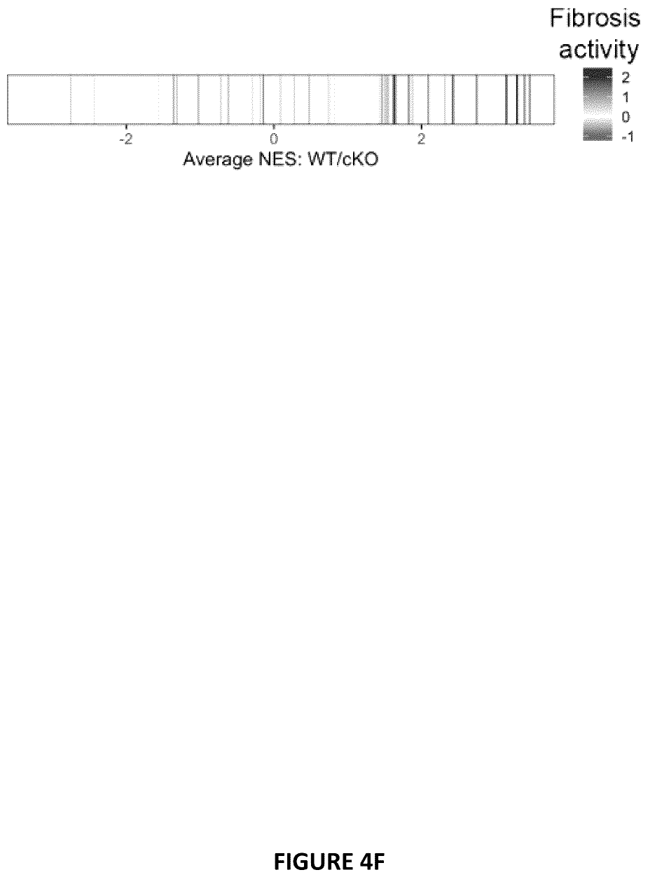

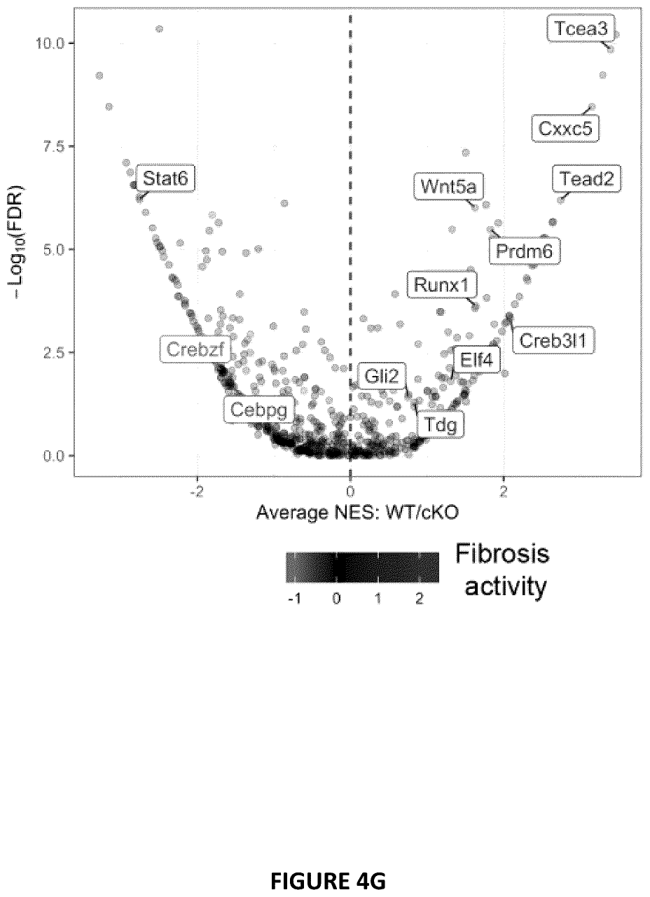

The present inventors next sought to examine the effect of Treg cell-derived Areg on hepatic stellate cell gene expression in NASH in vivo. Accordingly, the present inventors performed single cell RNA-Seq analysis of hepatic stellate cells enriched from livers of Treg cell-specific Areg conditional knockout (Areg^{Fl/Fl}Foxp3^{YFP-cre}) or littermate control (Areg^{Fl/Fl}) mice fed the CDAA-HFD. the present inventors observed four clusters of hepatic stellate cells, characterized by expression of: (1) Fcna (a marker of quiescent hepatic stellate cells), (2) Mmp2 and (3) Cxcl5 (markers of intermediately activated hepatic stellate cells), and (4) Acta2 (representing highly activated myofibroblast-like hepatic stellate cells) (FIG. 4A-4B). The Acta2 subset of hepatic stellate cells expressed Timp1 and were the only observed cell population to express the cytokine IL-6 (FIG. 4B).

Notably, the present inventors observed that Treg cell-derived Areg induced gene expression changes predominantly in the Fcna- and Mmp2-expressing clusters (FIG. 4C), consistent with Areg from Treg cells promoting the differentiation of more quiescent hepatic stellate cell populations into the highly activated, pro-fibrotic Acta2 subset. In the Fcna- and Mmp2-expressing clusters, Treg cell-derived Areg induced expression of genes encoding collagens and extracellular matrix-modifying enzymes and transcriptional signatures reflecting cellular proliferation (FIG. 4C-4D), confirming the role for Treg cell-derived Areg in driving the activation and expansion of hepatic stellate cells in the setting of NASH.

To identify transcription factors responsible for the transcriptional changes observed in hepatic stellate cells in response to Treg cell-derived Areg, the present inventors utilized the ARACNe-AP algorithm to reconstruct hepatic stellate cell transcriptional networks then incorporated these networks into msVIPER to score transcription factor activity within their hepatic stellate cell single cell RNA Seq dataset (FIG. 4E). Further, the present inventors utilized the ARACNe-AP output to generate a fibrosis activity score for each transcription factor, which quantifies their ability to regulate the expression of pro-fibrotic genes in hepatic stellate cells. the present inventors observed that transcription factors with a high fibrosis activity score had increased activity in hepatic stellate cells from Areg^{Fl/Fl} mice compared to Areg^{Fl/Fl}Foxp3^{YFP-cre} mice (FIG. 4F). Several transcription factors functionally shown to promote fibrotic gene expression in hepatic stellate cells had significantly greater activity in hepatic stellate cells from Areg^{Fl/Fl} mice compared to Areg^{Fl/Fl}Foxp3^{YFP-cre} mice (FIG. 4G), such as RUNX1 (that upregulates Timp155 and is thought to promote liver fibrosis), and CREB3L1, which was previously identified as a potential master regulator of the fibrotic response in hepatic stellate cells. Intriguingly, STAT6, which downregulates Il6 expression, had enhanced activity in hepatic stellate cells from Areg^{Fl/Fl}Foxp3^{YFP-cre} compared to Areg^{Fl/Fl} mice (FIG. 4G). Together, these data suggest that Treg cell-derived Areg is sensed by quiescent hepatic stellate cells in NASH and promotes activity of pro-fibrotic transcription factors that induce hepatic stellate cell activation and liver fibrosis.

### Example 15

### Treg Cell-Derived Areg Promotes Glucose Intolerance in a NASH-Dependent Manner Through EGFR Signaling on Hepatic Stellate Cells

NASH and insulin resistance are closely related manifestations of metabolic disease, with the interplay and causal relationship between the conditions difficult to distinguish. Treg cell immunosuppressive function has previously been shown to play a critical role in protecting HFD-fed mice (which do not progress to NASH) from developing insulin resistance; therefore, the present inventors sought to explore the potential contribution of Treg cell-derived Areg in the nexus of NASH and glucose intolerance using the FPC diet (which induces both NASH and insulin resistance).

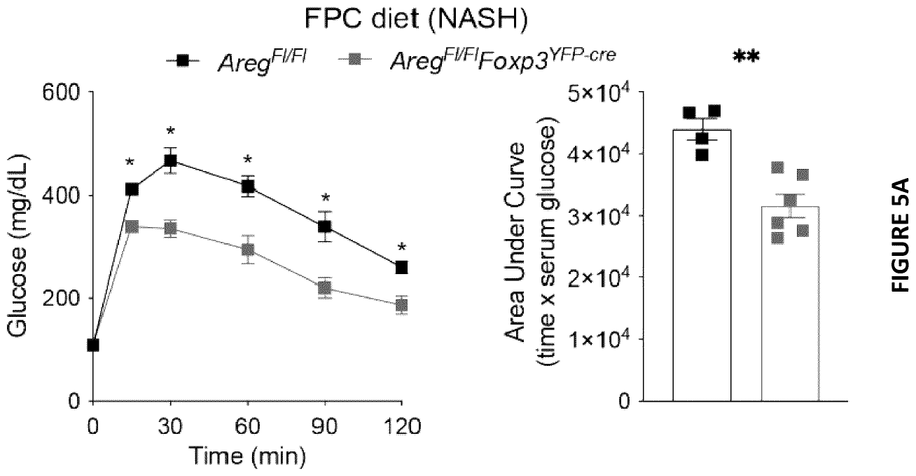

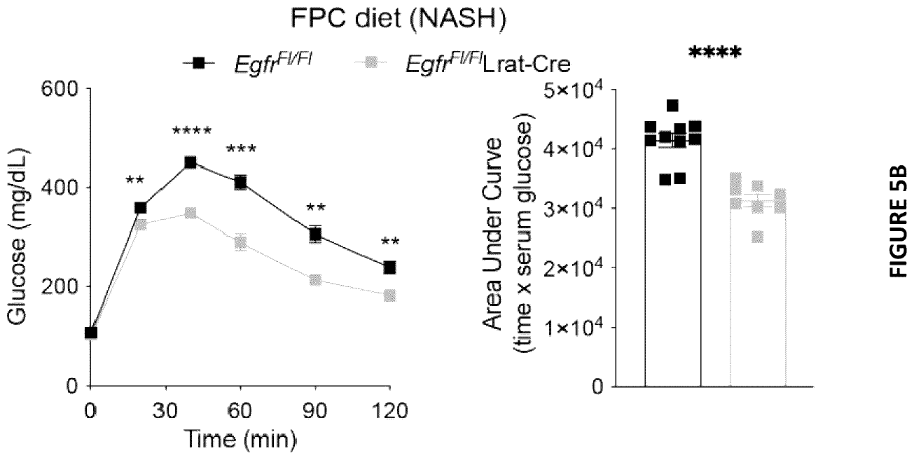

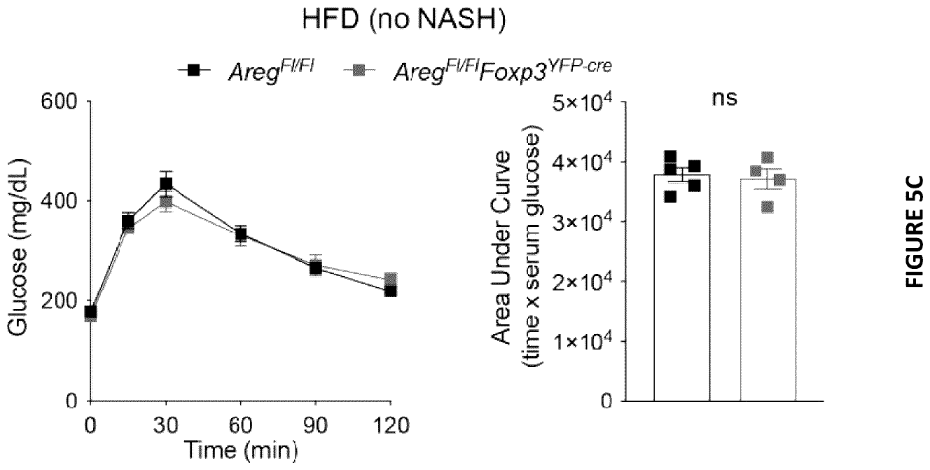

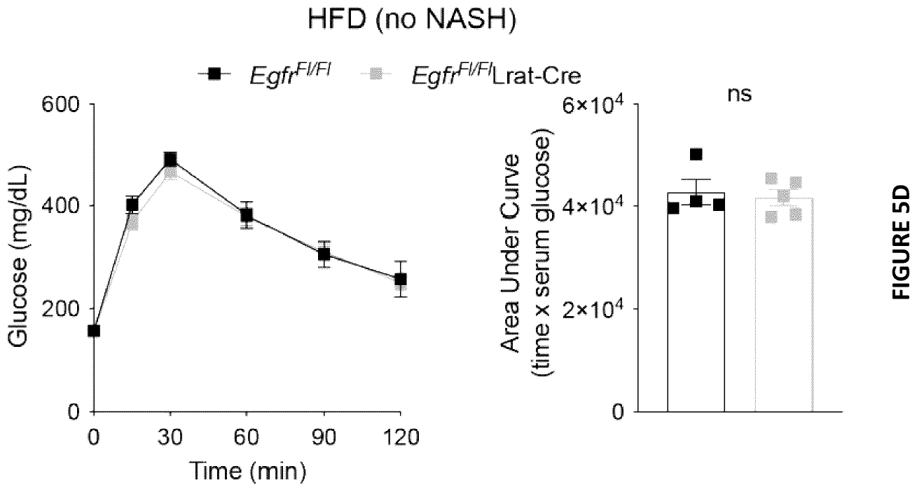

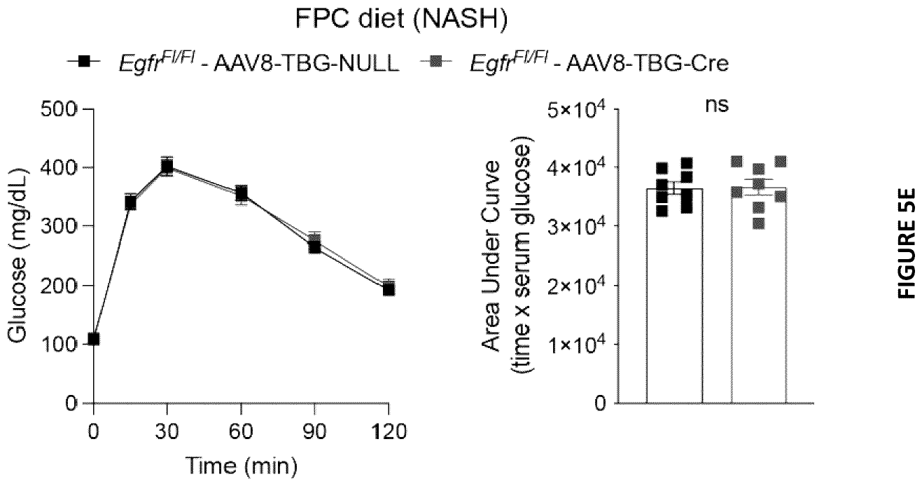

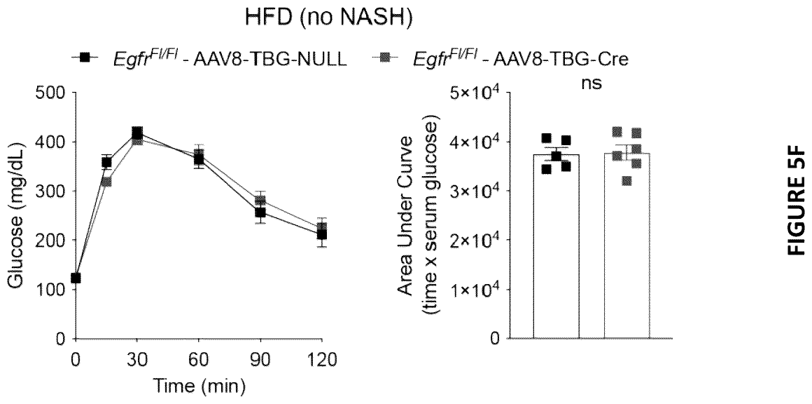

On the FPC diet, mice deficient for Areg production by all T cells (Areg^{Fl/Fl}CD4-Cre), or specifically by Treg cells (Areg^{Fl/Fl}Foxp3^{YFP-cre}), were significantly more glucose tolerant than corresponding littermate control (Areg^{Fl/Fl}) animals (FIG. 5A), suggesting Treg cell-derived Areg disrupts mechanisms of glucose uptake and/or metabolism in NASH. This protection in the absence of T cell- or Treg cell-derived Areg appeared to be liver-specific, as no differences in systemic glucose levels following injection of insulin were observed in the setting of FPC diet-induced NASH (data not shown), consistent with insulin primarily inducing glucose uptake in the fat and muscle, but not liver. As further evidence that a liver-specific mechanism may be responsible for the Areg-dependent glucose intolerance seen in NASH mice following glucose injection, no observable changes in fasting serum insulin levels were detected in the absence of either T cell-or Treg cell-derived Areg (data not shown).

Finally, in the setting of FPC diet-induced NASH, loss of EGFR on hepatic stellate cells in Egfr^{Fl/Fl}Lrat-Cre mice resulted in significantly greater glucose tolerance than in Egfr^{Fl/Fl} littermate controls (FIG. 5B), with no differences in systemic glucose levels observed following insulin injection (data not shown)—consistent with a role for Treg cell-derived Areg, acting via EGFR signaling on hepatic stellate cells, in promoting hepatic glucose intolerance. the present inventors next explored whether increased glucose tolerance in the absence of Treg cell-derived Areg also occurred in the absence of NASH, utilizing the standard HFD that induces insulin resistance systemically in the liver but does not induce NASH. Loss of Areg from T cells, including specifically Treg cells, or EGFR on hepatic stellate cells, did not affect glucose tolerance in HFD fed mice (FIG. 5C-5D), consistent with the observation that liver Treg cells fail to expand in this setting, which lacks liver damage and inflammation (data not shown). Furthermore, loss of Treg cell-derived Areg did not affect glucose sensitivity at an earlier timepoint (8 weeks) on the FPC diet (data not shown), when liver steatosis is evident but prior to the development of NASH, consistent with glucose intolerance being dependent on NASH and the expansion of Areg producing Treg cells and demonstrating that the observed phenotype is independent of FPC dietary components.

To examine whether Treg cell-derived Areg promotes glucose intolerance by directly signaling to hepatocytes, the present inventors specifically deleted Egfr in hepatocytes via tail vein injection of AAV8-TBG-Cre viral particles in adult Egfr^{Fl/Fl} mice, an approach that leads to >99% Cre mediated recombination specifically in hepatocytes (data not shown). Notably, deletion of Egfr in hepatocytes did not alter glucose tolerance with or without NASH (FIG. 5E-5F) and did not affect NASH-induced liver fibrosis (data not shown). Although EGFR signaling on hepatocytes is essential to liver regeneration, these data suggest EGFR signaling on hepatic stellate cells, not hepatocytes, promotes liver fibrosis and glucose intolerance in NASH. Taken together, these data demonstrate that Treg cell-derived Areg, via EGFR-mediated activation of hepatic stellate cells, promotes glucose intolerance in a NASH-dependent manner, in contrast to previous studies which utilized Treg cell-transfer and-depletion models to demonstrate a protective role for Treg cells in preventing insulin resistance in the setting of HFD feeding.

### Example 16

### Treg Cell-Derived Areg Promotes Hepatocyte Gluconeogenesis in NASH Through Hepatic Stellate Cell-Derived IL-6

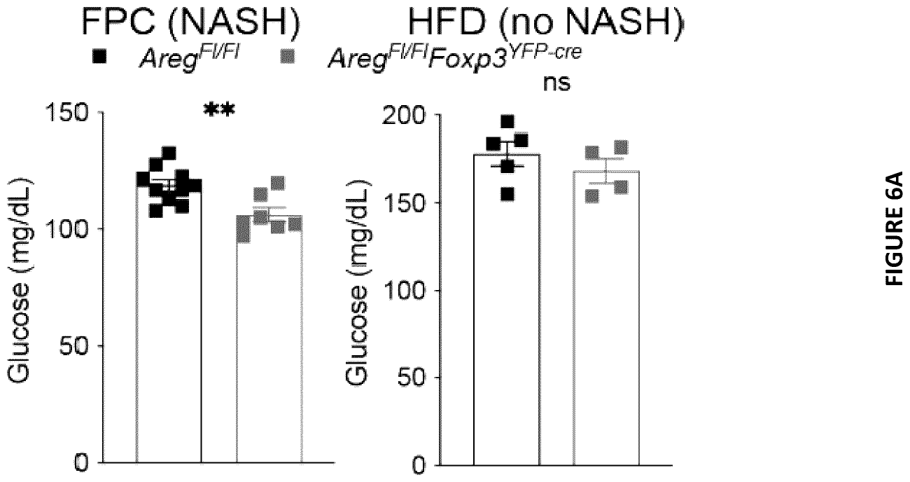

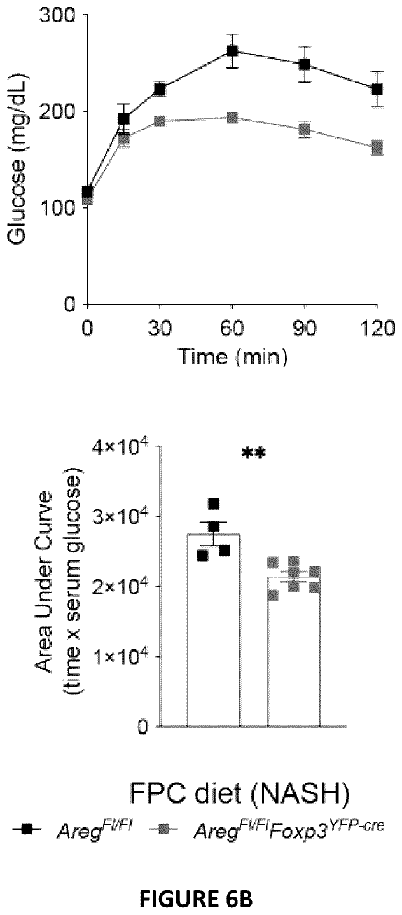

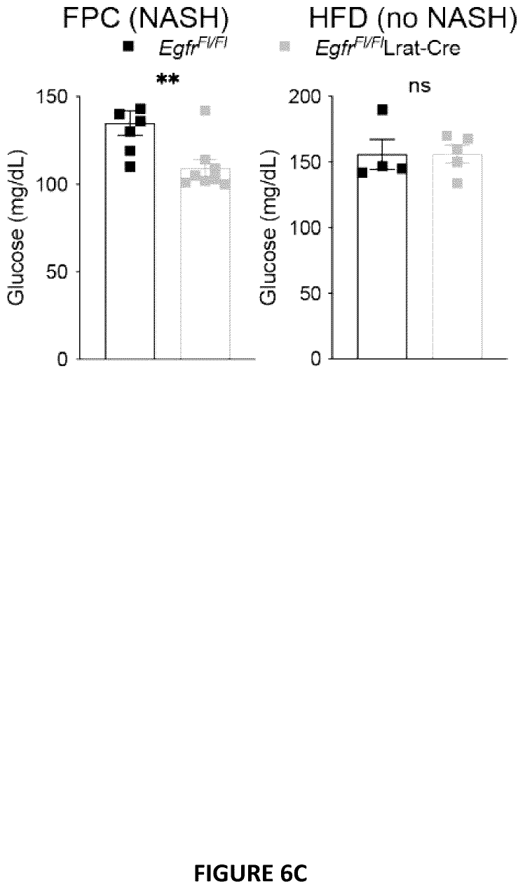

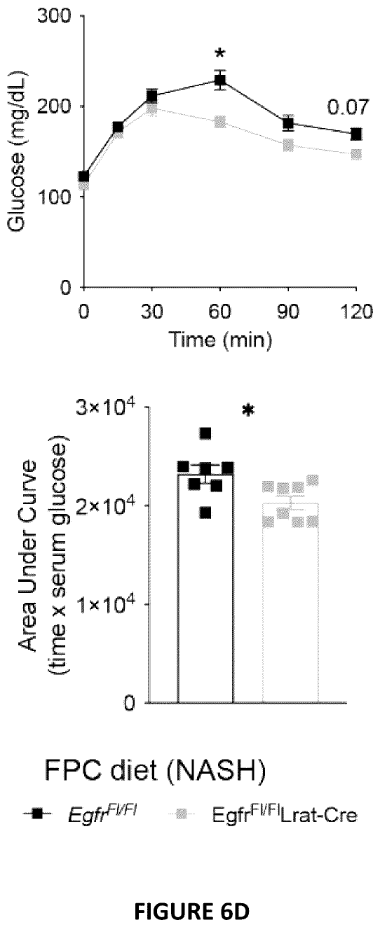

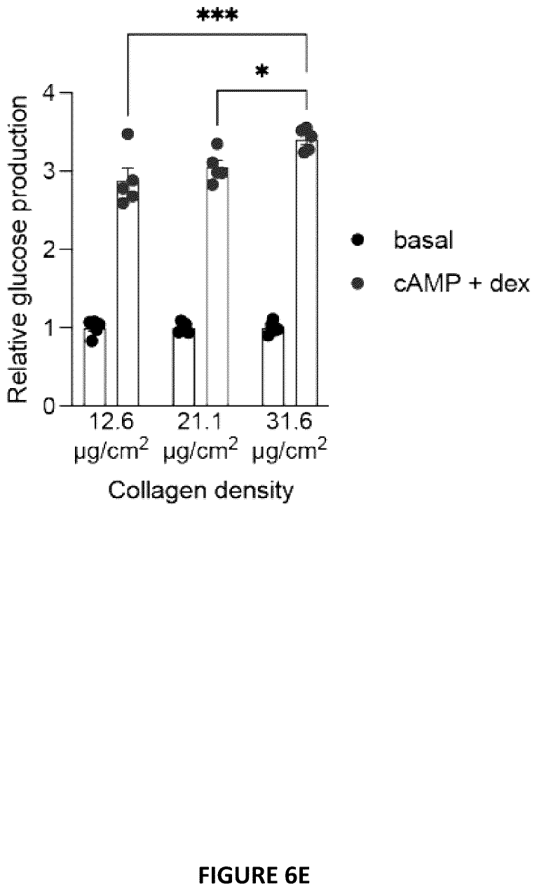

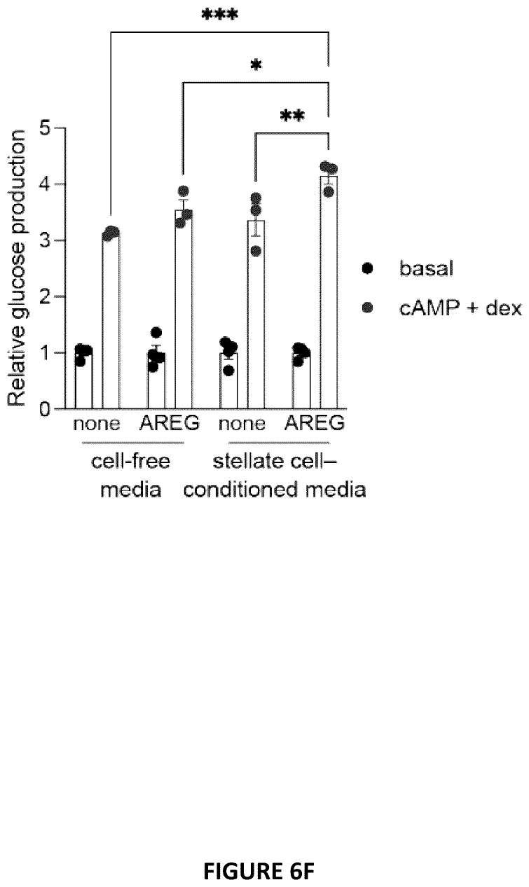

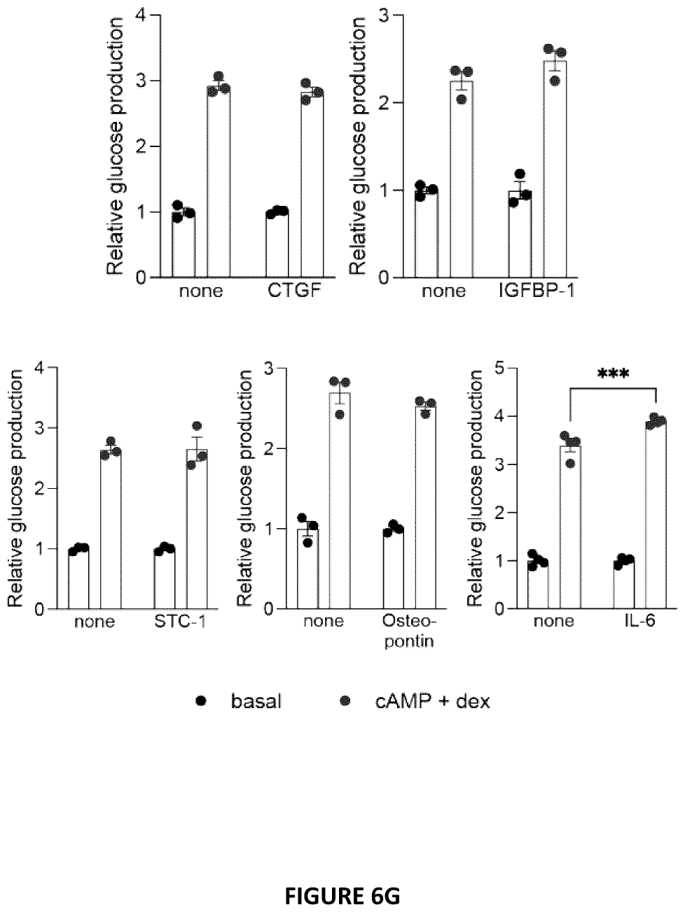

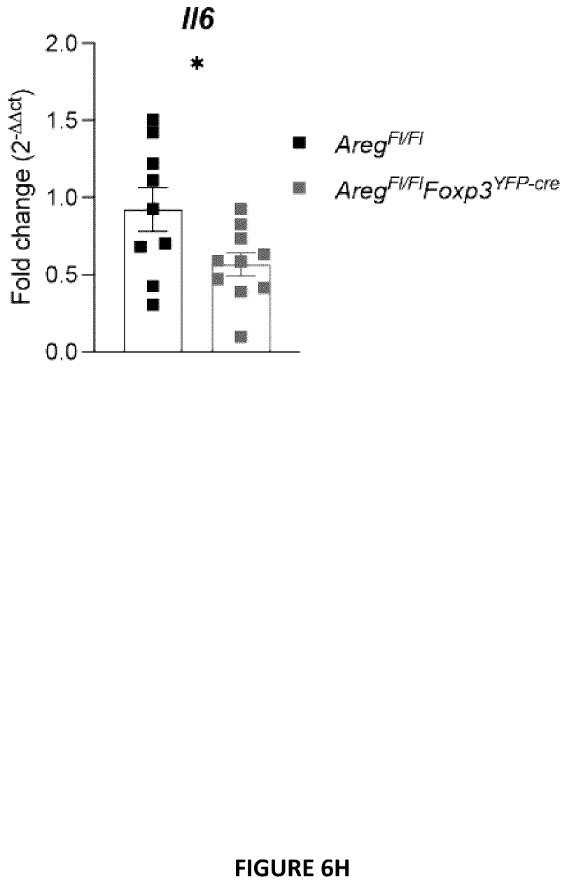

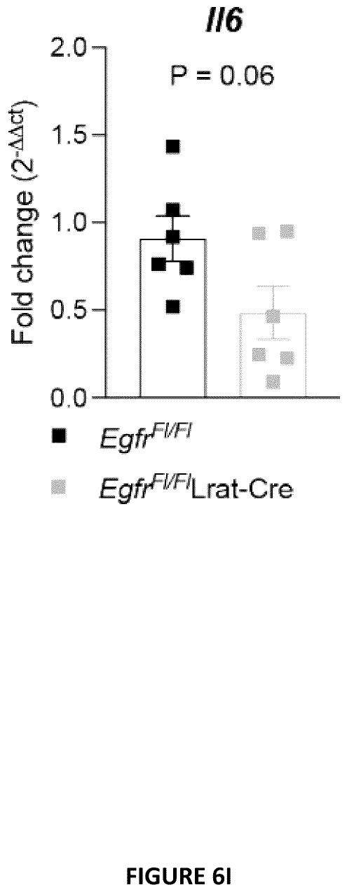

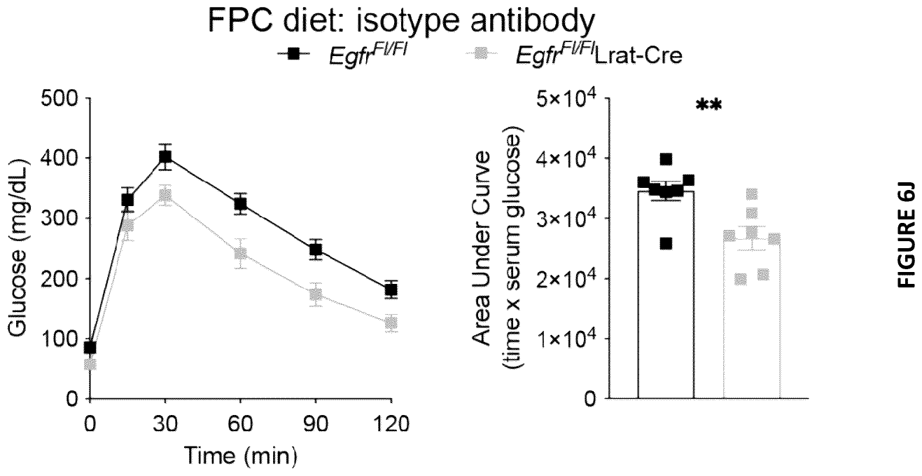

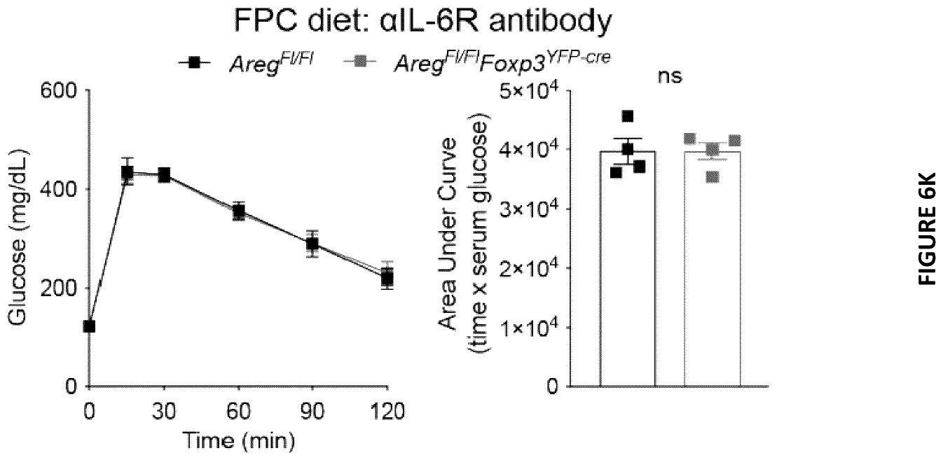

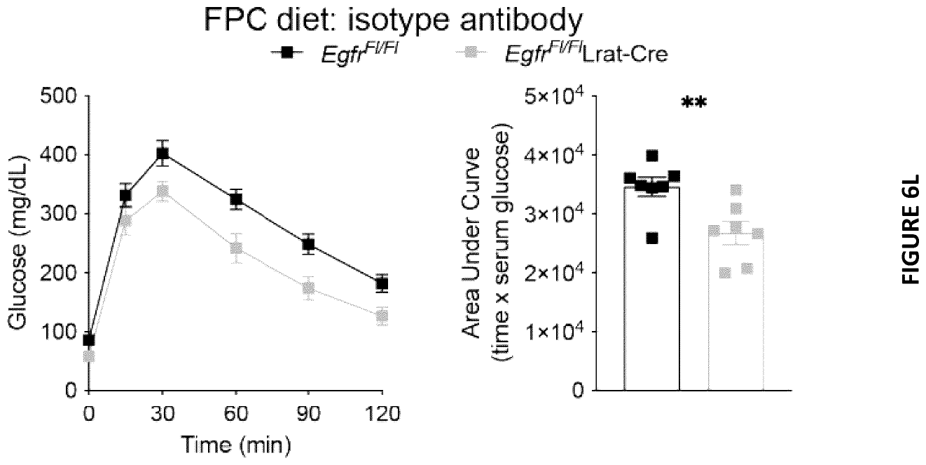

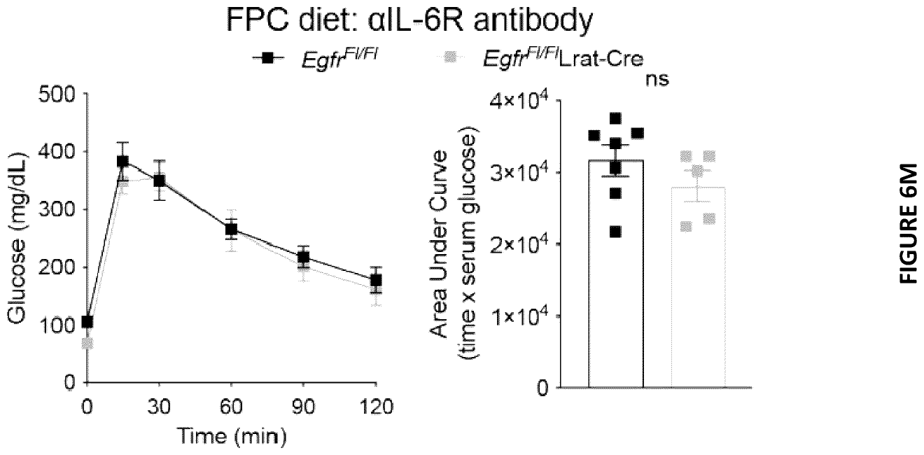

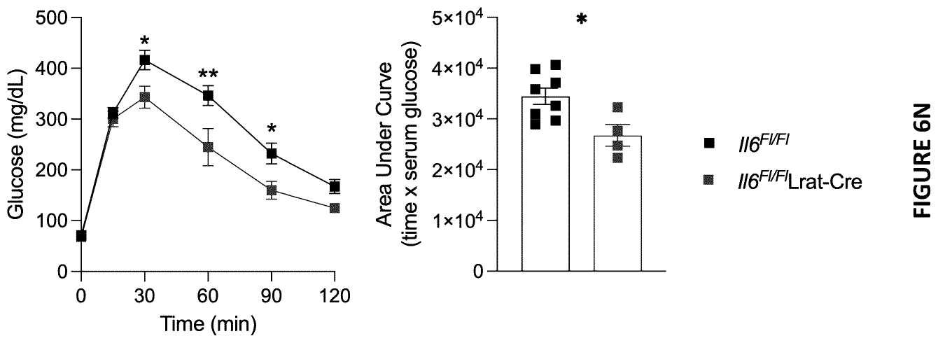

To study how Treg cell-derived Areg contributes to glucose intolerance, the present inventors considered the role of the liver in glucose homeostasis; specifically, that the liver serves as a key site of gluconeogenesis, a major therapeutic target in insulin resistance. In mice fed the FPC diet, but not the HFD, the present inventors observed reduced fasting blood glucose levels in the absence of Treg cell-derived Areg (FIG. 6A), suggesting Treg cell-derived Areg may promote gluconeogenesis in a NASH dependent manner. For functional assessment of hepatic gluconeogenesis in NASH, a pyruvate tolerance test (PTT) was performed in FPC diet-fed mice. In this setting, loss of Treg cell-derived Areg led to reduced systemic blood glucose levels following pyruvate injection (FIG. 6B). Moreover, deletion of Egfr in hepatic stellate cells led to reduced gluconeogenesis in NASH, as assessed by fasting blood glucose levels (FIG. 6C) and the PTT (FIG. 6D), which, in sum, is consistent with Treg cell-derived Areg promoting hepatic gluconeogenesis in a NASH-dependent manner via EGFR signaling-mediated activation of hepatic stellate cells.

The present inventors next sought to investigate how Treg cell-derived Areg promotes hepatic gluconeogenesis in NASH. To this end, the present inventors first examined whether tissue fibrosis influences hepatocyte glucose production, hypothesizing that Treg cell-derived Areg could contribute to hepatocyte gluconeogenesis indirectly via promoting NASH-mediated fibrosis by activation of hepatic stellate cells (FIG. 1, 3). Using an in vitro primary mouse hepatocyte glucose production assay hepatocytes cultured on higher collagen density had significantly greater glucose production (FIG. 6E), suggesting Treg cell-derived Areg may promote hepatic gluconeogenesis by increasing the extent of liver fibrosis in the setting of NASH (FIG. 1G-1K). Alternatively, Treg cell-derived Areg could promote the production of a soluble mediator by hepatic stellate cells that directly influences hepatocyte glucose production.

To examine this possibility, the present inventors adapted the hepatocyte glucose production assay to treat hepatocytes with media conditioned by Areg-409 stimulated hepatic stellate cells prior to assessing glucose production. Using this approach, the present inventors observed increased glucose production with media conditioned by Areg-treated hepatic stellate cells relative to cell free media controls (with or without the addition of Areg), and media conditioned by hepatic stellate cells without Areg stimulation (FIG. 6F), suggesting Areg promotes the production of soluble factor(s) by hepatic stellate cells that stimulate hepatocyte glucose production.

The present inventors then focused on screening potential hepatic stellate cell-derived mediators that were upregulated in response to Areg stimulation (FIG. 3E), screening hepatic stellate cell-derived molecules by treating hepatocytes with corresponding recombinant versions of each candidate. Primary murine hepatocytes treated with recombinant IL-6 produced significantly greater glucose (FIG. 6G), consistent with previous studies that indicated IL-6 promotes hepatic insulin resistance through IL-6/STAT3-induced inhibition of insulin receptor signaling, and work demonstrating that IL-6 receptor signaling on hepatocytes promotes hepatic gluconeogenesis in mouse models of acute stress. Notably, several other candidate mediators (FIG. 3E), including connective tissue growth factor (CTGF/CCN2), stanniocalcin-1 (STC-1), osteopontin, and insulin like growth factor binding protein 1 (IGFBP-1) did not affect glucose production when added directly to hepatocyte cultures (FIG. 6G).

The present inventors next interrogated whether Treg cell-derived Areg promotes glucose intolerance through IL-6 in vivo. the present inventors observed reduced expression of Il6 in the livers of mice lacking Treg cell-derived Areg compared to littermate Areg^{Fl/Fl} controls when fed the FPC diet (FIG. 6H). Similarly, Il6 expression in mice with hepatic stellate cell specific Egfr deletion was reduced compared to Egfr^{Fl/Fl} littermate controls in liver tissue from mice fed the FPC diet (FIG. 6I). Previously, the present inventors observed Il6 expression solely by activated hepatic stellate cells in NASH (FIG. 4B), and enhanced activity of STAT6—known to downregulate Il6 expression-in hepatic stellate cells from NASH mice lacking Treg cell—derived Areg compared to controls (FIG. 4G). These findings are consistent with Treg cell—derived Areg, and EGFR-signaling on hepatic stellate cells, promoting Il6 expression by hepatic stellate cells in NASH.

To functionally assess whether Treg cell-derived Areg promotes glucose intolerance via IL-6 in vivo, the present inventors blocked IL-6/IL-6R signaling prior to the glucose tolerance testing in mice on the FPC diet. Injection of an antibody that blocks IL-6/IL-6R signaling, but not an isotype control antibody, restored glucose tolerance in Areg^{Fl/Fl} mice to levels similar as those lacking Treg cell-derived Areg (Areg^{Fl/Fl}Foxp3^{YFP-cre}) (FIG. 6J-6K), consistent with IL-6R signaling being responsible for the observed glucose intolerance in the setting of FPC diet-induced NASH. Further, blockade of IL-6/IL-6R signaling restored glucose tolerance in Egfr^{Fl/Fl} mice to levels similar as those lacking EGFR on hepatic stellate cells (Egfr^{Fl/Fl}Lrat-Cre) (FIG. 6L-6M). Finally, the present inventors specifically deleted Il6 in hepatic stellate cells. Loss of Il6 in hepatic stellate cells resulted in improved glucose tolerance in Il6^{Fl/Fl}Lrat-Cre mice compared to littermate Il6^{Fl/Fl} mice (FIG. 6N). Taken together, these data support a mechanism whereby Treg cell-derived Areg drives hepatic glucose intolerance through EGFR-mediated activation of hepatic stellate cells-exacerbating liver fibrosis and increasing production of hepatic stellate cell-derived IL-6-which in turn stimulate hepatocyte gluconeogenesis and raise serum glucose levels.

While this invention has been disclosed with reference to particular embodiments, it is apparent that other embodiments and variations of the inventions disclosed herein can be devised by others skilled in the art without departing from the true spirit and scope thereof. The appended claims include all such embodiments and equivalent variations.

What is claimed is:

1. A method of treating a subject with non-alcoholic steatohepatitis (NASH) comprising administering to a subject a therapeutically effective amount of an adeno-associated virus vector comprising a nucleic acid that results in
(a) a mutation or deletion of a gene encoding an epidermal growth factor receptor expressed in hepatic stellate cells in the subject; or
(b) a mutation or deletion of a gene encoding an interleukin-6 receptor expressed in hepatocytes in the subject; or
(c) a mutation or deletion of a gene encoding interleukin-6 expressed in hepatic stellate cells in the subject.

2. A method of treating a subject with non-alcoholic steatohepatitis (NASH) comprising administering to a subject a therapeutically effective amount of one or more anti-epidermal growth factor inhibitors.

3. The method of claim 2, wherein the anti-epidermal growth factor inhibitor is a small molecule tyrosine kinase inhibitor (TKI).

4. The method of claim 3, wherein the TKI is selected from the group consisting of afatinib, dacomitinib, neratinib, osimertinib, brigatinib, erlotinib, geftinib, lapatinib, mobocertinib, tivozanib, pemetrexed, and vandetanib.

5. The method of claim 2, wherein the anti-epidermal growth factor inhibitor is an antibody.

6. The method of claim 5, wherein the antibody is selected from the group consisting of cetuximab, panitumumab, and necitumumab.

7. A method of treating a subject with non-alcoholic steatohepatitis (NASH) comprising administering to a subject a therapeutically effective amount of one or more anti-interleukin-6 receptor inhibitors.

8. The method of claim 7, wherein the interleukin-6 receptor inhibitor is an antibody.

9. The method of claim 8, wherein the selected from the group consisting of sarilumab, tocilizumab, and satralizumab.

10. A method of treating a subject with non-alcoholic steatohepatitis (NASH) comprising administering to a subject a therapeutically effective amount of one or more anti-interleukin-6 inhibitors.

11. The method of claim 10, wherein the interleukin-6 inhibitor is a kinase inhibitor.

12. The method of claim 11, wherein the kinase inhibitor is tofacitinib.

13. The method of claim 10, wherein the interleukin-6 inhibitor is an antibody.

14. The method of claim 13, wherein the antibody is siltuximab.
